(19) United States
(12) Patent Application Publication
(10) Pub. No.: US 2016/0022895 A1
(43) Pub. Date: Jan. 28, 2016

(54) STOCK SOLUTION CONCENTRATING DEVICE, STOCK SOLUTION TREATMENT DEVICE, AND CIRCULATION-TYPE TREATMENT DEVICE

(71) Applicants: TOKUSHIMA UNIVERSITY, Tokushima-shi, Tokushima (JP); Takatori Corporation, Kashihara-shi, Nara (JP)

(72) Inventors: Toshiya OKAHISA, Tokushima-shi, Tokushima (JP); Yohsuke KINOUCHI, Tokushima-shi, Tokushima (JP); Tadahiko NAKAGAWA, Tokushima-shi, Tokushima (JP); Satoru YAMAJI, Kashihara-shi, Nara (JP); Zenji TANAKA, Kashihara-shi, Nara (JP)

(73) Assignees: TAKATORI CORPORATION, Kashihara-shi, Nara (JP); TOKUSHIMA UNIVERSITY, Tokushima-shi, Tokushima (JP)

(21) Appl. No.: 14/761,226

(22) PCT Filed: Jan. 14, 2014
(86) PCT No.: PCT/JP2014/000115

§ 371 Date: Jul. 15, 2015

(30) Foreign Application Priority Data
Jan. 15, 2013 (JP) 2013-004791
May 16, 2013 (JP) 2013-104363

(51) Int. Cl. A61M 1/34; B01D 69/08

(52) U.S. Cl. CPC A61M 1/3486; B01D 69/08

(57) ABSTRACT

Provided are a stock solution concentrating device, a stock solution treatment device and a circulation-type treatment device that can prevent the deposition of cells and the like on a filtration member and that can continuously filter and concentrate a stock solution such as pleural and ascitic fluid or blood plasma. The stock solution concentrating device concentrates a stock solution such as pleural and ascitic fluid or blood plasma to form a concentrated solution, and is equipped with: a filter (10) having a filtration member that filters the stock solution; a concentrator (20) to which the filtrate which has been filtered is supplied, and which concentrates the filtrate to form a concentrated solution; and a stock solution supply unit that supplies the stock solution to the filter (10). The stock solution supply unit has a supply amount adjustment function for adjusting the amount of the stock solution supplied to the filter.

This application is a U.S. National Phase under 35 U.S.C. §371 of International Application PCT/JP2014/000115, filed on Jan. 14, 2014, which claims priority to Japanese Patent Application No. 2013-004791 filed on Jan. 15, 2013, and to Japanese Patent Application No. 2013-104363, filed on May 16, 2013. All publications, patents, patent applications, databases and other references cited in this application, all related applications referenced herein, and all references cited therein, are incorporated by reference in their entirety as if restated here in full and as if each individual publication, patent, patent application, database or other reference were specifically and individually indicated to be incorporated by reference.

### TECHNICAL FIELD

The present invention relates to a stock solution concentrating device, a stock solution treatment device, and a circulation-type treatment device. More particularly, the present invention relates to a stock solution concentrating device for obtaining a treatment solution by filtering and concentrating a stock solution such as pleural and ascitic fluid accumulated in the thorax or abdomen, caused by carcinomatous pleurisy and hepatic cirrhosis or plasma waste fluid discarded in plasma exchange, which is administered by intravenous infusion, a stock solution treatment device including the stock solution concentrating device, and a circulation-type treatment device for extracorporeal circulation of the pleural and ascitic fluid.

### BACKGROUND ART

In carcinomatous pleurisy and hepatic cirrhosis, pleural fluid or ascitic fluid is sometimes accumulated in the thoracic and peritoneal cavities. The accumulated state of the pleural and ascitic fluid presses on nearby organs. In order to solve such a problem, fine-needle aspiration of the pleural and ascitic fluid may be performed.

On the other hand, the pleural and ascitic fluid contains some or all of plasma components leaked from the blood. Major proteins (e.g., albumin and globulin) are contained in the blood plasma. Accordingly, although the above condition is improved by aspiration of the pleural and ascitic fluid, water as well as components useful for human body such as proteins are lost. The lost components need to be supplemented by the intravenous administration of albumin and globulin preparations.

However, although a certain component can be supplied by the intravenous administration of albumin preparation, the preparation is expensive and the cost of medical treatment is very high.

In addition, only a certain component is supplied, and thus only a partial component of the lost components is supplemented. This may cause problems such as undernutrition and increased susceptibility to infection.

There has been developed a method comprising the steps of: treating the pleural fluid or ascitic fluid, which has been aspirated from the thoracic or peritoneal cavity; and intravenously administering the resultant fluid, a so-called cell-free and concentrated ascites reinfusion therapy (CART). In the CART, the pleural fluid or ascitic fluid is supplied from a bag (stock solution bag) containing the collected pleural fluid or ascitic fluid to a filter having a hollow fiber membrane so as to separate a liquid component. The separated liquid component is concentrated by removing some of water through a concentrator. The obtained concentrated solution is intravenously administered. In the case of the CART, a large part of an active component except a cell component, contained in the pleural fluid or ascitic fluid can be returned to the patient's body. Thus, the component lost from the blood can be effectively supplied to the patient without limitation to a certain component. Additionally, even if the concentrated solution is administered, an insufficient amount of the component may be supplemented using a preparation having an amount corresponding to the insufficient amount. The amount of the albumin preparation can be reduced as much as possible, resulting in a reduction in the cost of medical treatment.

Incidentally, in the case where a concentrated solution used for CART is currently produced, a stock solution (pleural fluid or ascitic fluid) to be treated is supplied to the filter and the concentrator. As the method for supplying the pleural fluid or ascitic fluid, there has been developed a technology of using a gravity method (drop system) and a mechanical supplying method (pump system) (refer to Patent Documents 1 and 2).

The drop system is a method comprising the steps of: connecting a stock solution bag, a filter, and a concentrator in this order to a tube; disposing these components so that the height is set lower in this order; and supplying the pleural and ascitic fluid in the stock solution bag to the filter and the concentrator by gravity.

However, although the drop system is advantageous in that a special transport device is not necessary because the stock solution is flowed into the filter and the concentrator only by the gravity, it is not possible to largely increase the flow rate (supply flow rate) for supplying the stock solution to the concentrator. This causes a problem of requiring a long time for the treatment.

Further, the concentration of the concentrated solution varies depending on the supply flow rate and the flow rate of the water discharged from the concentrator (discharge flow rate). In addition, the supply flow rate varies depending on not only the difference in the height of each device but also the discharge flow rate. For this reason, in order to obtain a concentrated solution having a desired concentration, fine adjustment is needed for the arrangement of each device and each discharge flow rate. There is a problem such that it is difficult to adjust the concentration of the concentrated solution.

On the other hand, the pump system is a method comprising the steps of: forming a pump such as a roller pump on a tube for connecting a stock solution bag to a filter; and pumping a stock solution so as to supply it to the filter and the concentrator. For this reason, although an extra pump is needed as compared to the drop system, the treatment time can be reduced and it is easy to keep the supply flow rate constant because the stock solution is mechanically flowed. This results in an advantage of easily adjusting the concentration of the concentrated solution. Hence, the use of the pump system enables the

### PRIOR ART DOCUMENTS

### Patent Documents

Patent Document 1: JP-A-2009-284936

Patent Document 2: JP-A-2012-125557

### SUMMARY OF THE INVENTION

### Problems to be Solved by the Invention

However, in each case of the drop system and the pump system, the filter uses a filtration method for trapping cells in a stock solution by a hollow fiber membrane. As the filtration treatment is performed, the hollow fiber membrane may be clogged by the trapped cells. This increases the pressure damage on the filter, whereby the volume of the liquid passing through the hollow fiber membrane is reduced and the treatment efficiency is decreased. When it is impossible to perform the filtration, the hollow fiber membrane has to be cleaned.

Patent Document 2 discloses that clogging can be prevented by using an ultrafilter having ultrafiltration performance which is suitable for ascitic fluid. However, the deposition of cells with the progression of the treatment causes clogging. Further, Patent Document 2 does not describe any method for preventing the deposition of the cells.

Under the circumstances, an object of the present invention is to provide a stock solution concentrating device that can prevent cells from depositing on a filtration member, and continuously treat a stock solution such as pleural and ascitic fluid or blood plasma.

Further, another object of the present invention is to provide a stock solution treatment device including the stock solution concentrating device that can continuously treat the stock solution.

Further, another object of the present invention is to provide a circulation-type treatment device for extracorporeal circulation of the stock solution such as pleural and ascitic fluid or blood plasma.

### Means for Solving the Problems

### (Stock Solution Concentrating Device)

According to a 1st aspect of the invention, there is provided a stock solution concentrating device which is a stock solution concentrating device for concentrating a stock solution such as pleural and ascitic fluid or blood plasma to form a concentrated solution, the device comprising: a filter having a filtration member that filters the stock solution; a concentrator to which the filtrate which has been filtered is supplied, and which concentrates the filtrate to form the concentrated solution; and a stock solution supply unit that supplies the stock solution to the filter, wherein the filter includes a supply port to which the stock solution is supplied and a separate liquid discharge port to which a separate liquid which has been separated from the filtrate is discharged, and the stock solution supply unit has a supply amount adjustment function for adjusting the amount of the stock solution supplied to the filter the filter includes a supply port to which the stock solution is supplied, and a separate liquid discharge port from which a separate liquid which has been separated from the filtrate is discharged, the stock solution supply unit includes a flow path that supplies the stock solution to the supply port of the filter, and a circulation flow path that supplies the separate liquid discharged from the separate liquid discharge port of the filter to the supply port of the filter, and the circulation flow path is equipped with a circulation flow forming means that forms a flow of the separate liquid flowing from the separate liquid discharge port of the filter to the supply port of the filter.

According to a 2nd aspect of the invention, there is provided a stock solution concentrating device for concentrating a stock solution such as pleural and ascitic fluid or blood plasma to form a concentrated solution, the device comprising: a filter having a filtration member that filters the stock solution; a concentrator to which a filtrate which has been filtered by the filter is supplied, and which concentrates the filtrate to form the concentrated solution; and a stock solution supply unit that supplies the stock solution to the filter, wherein the stock solution supply unit has a supply amount adjustment function for adjusting the amount of the stock solution supplied to the filter, the filter includes a supply port to which the stock solution is supplied, and a separate liquid discharge port from which a separate liquid which has been separated from the filtrate is discharged, the stock solution supply unit includes a circulation flow path that supplies the separate liquid discharged from the separate liquid discharge port of the filter to the supply port of the filter, and the circulation flow path is equipped with a circulation flow forming means that forms a flow of the separate liquid flowing from the separate liquid discharge port of the filter to the supply port of the filter, the circulation flow forming means includes a nozzle whose end is connected to the circulation flow path and a stock solution supply means that supplies the stock solution to the nozzle, the end of the nozzle is connected to the circulation flow path so that the stock solution is supplied from the end of the nozzle to the circulation flow path when the stock solution is supplied from the stock solution supply means, and the nozzle is disposed so that a flow of the stock solution having a speed component in a direction flowing from the separate liquid discharge port of the filter to the supply port of the filter is formed by the stock solution supplied from the end of the nozzle to the circulation flow path.

According to a 3rd aspect of the invention, there is provided the stock solution concentrating device of the 2nd aspect, wherein the stock solution supply unit includes wherein the stock solution supply unit includes a flow generating means that forms a flow flowing from the filter to the concentrator on a flow path that allows the filter to be communicated with the concentrator.

According to a 4th aspect of the invention, there is provided the stock solution concentrating device of the 1^{St} aspect, wherein the circulation flow forming means includes a nozzle whose end is connected to the circulation flow path and a stock solution supply means that supplies the stock solution to the nozzle, the end of the nozzle is connected to the circulation flow path so that the stock solution is supplied from the end of the nozzle to the circulation flow path when the stock solution is supplied from the stock solution supply means, the nozzle is disposed so that a flow of the stock solution having a speed component in a direction flowing from the separate liquid discharge port of the filter to the supply port of the filter is formed by the stock solution supplied from the end of the nozzle to the circulation flow path.

According to a 5th aspect of the invention, there is provided the stock solution concentrating device of the 2^{nd} aspect, wherein the stock solution supply means includes a stock solution storage part for storing the stock solution that is communicated with the nozzle and a pressurization mechanism that pressurizes the stock solution in the stock solution storage part.

According to a 6th aspect of the invention, there is provided the stock solution concentrating device of the 5th aspect, wherein a water removing means that discharges water separated from the filtrate from the concentrator at a predetermined flow rate, wherein the pressurization mechanism includes a pressurizing force controller that detects a flow rate of the concentrated solution discharged from the concentrator and adjusts the pressurizing force pressurizing the stock solution based on the flow rate of the concentrated solution.

According to a 7th aspect of the invention, there is provided the stock solution concentrating device of the 5th aspect, comprising a concentrated solution discharge means that discharges the concentrated solution from the concentrator at a predetermined flow rate, wherein the pressurization mechanism includes a pressurizing force controller that detects the flow rate of water separated from the filtrate discharged from the concentrator and adjusts the pressurizing force pressurizing the stock solution based on the flow rate of the water.

According to an 8th aspect of the invention, there is provided the stock solution concentrating device of the 5th aspect, wherein the stock solution supply means includes a weight measurement function that measures the weight of the stock solution stored in the stock solution storage part, the pressurization mechanism includes a water weight measurement unit that measures the weight of water separated from the filtrate and a concentrated solution weight measurement unit that measures the weight of the concentrated solution discharged from the concentrator, the pressurization mechanism includes a pressurizing force controller that adjusts the pressurizing force pressurizing the stock solution based on all or any of the weight of the stock solution measured by the weight measurement function of the stock solution supply means, the weight of the concentrated solution measured by the concentrated solution weight measurement unit, and the weight of the water measured by the water weight measurement unit.

According to a 9th aspect of the invention, there is provided the stock solution concentrating device of any of the 1st or 2nd aspect, wherein a separated product recovery unit that discharges a liquid and/or residue in the circulation flow path and the filter is provided on the circulation flow path.

According to a 10th aspect of the invention, there is provided the stock solution concentrating device of any of the 1st or 2nd aspect, wherein the concentrator includes a concentrated solution recovery pipe that is connected to the separate liquid discharge port, and the concentrated solution recovery pipe includes a plurality of branch pipelines to which a concentrated solution storage container for storing a concentrated solution is attached.

According to an 11th aspect of the invention, there is provided the stock solution concentrating device of the 10th aspect, wherein the plurality of branch pipelines are provided so as to form a difference in the height of the concentrated solution storage containers.

According to a 12th aspect of the invention, there is provided the stock solution concentrating device of the 10^{th} aspect, wherein a flow rate adjusting members that be capable of establishing or blocking the communication between each of the concentrated solution bags and the concentrator is provided on the plurality of branch pipelines.

According to a 13th aspect of the invention, there is provided the stock solution concentrating device of the 1st or 2nd aspects, comprising a heating means that heats the stock solution and/or filtrate.

### (Stock Solution Treatment Device)

According to a 14th aspect of the invention, there is provided a stock solution treatment device that recovers a stock solution such as pleural and ascitic fluid or blood plasma, the device comprising: the stock solution concentrating device according to the first or 2nd aspects; and a recovery unit that recovers a stock solution and supplies the recovered stock solution to the filter of the ascitic fluid concentration device, wherein the concentrator of the stock solution concentrating device includes a concentrated solution recovery pipe that is connected to the separate liquid discharge port, and the concentrated solution recovery pipe includes a plurality of branch pipelines to which concentrated solution storage containers for storing a concentrated solution are attached.

According to a 15th aspect of the invention, there is provided a stock solution treatment device of the 14th aspect, wherein the recovery unit includes a pair of bags for storing the stock solution, and further includes a flow path adjusting mechanism that alternately supplies the stock solution to the pair of bags, and supplies the stock solution from one bag of the pair of bags to which a stock solution is not supplied, to the filter of the ascitic fluid concentration device.

According to a 16th aspect of the invention, there is provided the stock solution treatment device of the 14th aspect, wherein the recovery unit includes a recovery sheet having a plurality of recovery chambers isolated from each other, in each of the recovery chambers of the recovery sheet, a stock solution supply port for supplying a stock solution from the outside to the recovery chamber, a stock solution discharge port for discharging the stock solution in the recovery chamber to the outside, and a separation part that separates the inside of the recovery chamber into a front space communicated with the stock solution supply port and a rear space communicated with the stock solution discharge port are provided, and the separation part is formed so that the front space is communicated with the rear space when the stock solution in the front space is pressurized in a state where the stock solution is stored in the front space.

According to a 17th aspect of the invention, there is provided the stock solution treatment device of the 16th aspect, wherein the recovery unit includes a winding unit that winds the recovery sheet and a pressurization unit that is disposed at the upstream of the winding unit, and the pressurization unit sequentially pressurizes the recovery sheet wound by the winding unit.

### (Circulation Treatment Device)

According to an 18th aspect of the invention, there is provided a circulation-type treatment device that recovers a stock solution such as pleural and ascitic fluid or blood plasma from the living body, performs an extracorporeal treatment, and returns the stock solution to the living body, the device comprising: a recovery unit that recovers the stock solution discharged from the living body; a treatment unit that treats the stock solution recovered by the recovery unit; and a return unit that returns the treatment solution treated by the treatment unit to the living body, wherein the recovery unit includes a reservoir that once stores the stock solution discharged from the living body, the treatment unit is the stock solution treatment device according to the 14th aspect.

According to a 19th aspect of the invention, there is provided the circulation-type treatment device of the 18th aspect, wherein a recovery tube that allows the living body to be communicated with a reservoir is provided on the recovery unit, and a sensor that detects a state of the stock solution flowing in the recovery tube is provided on the recovery tube.

According to a 20th aspect of the invention, there is provided the circulation-type treatment device of the 18th aspect, the treatment unit and the return unit are connected to each other and stored in a case.

According to a 21st aspect of the invention, there is provided the circulation-type treatment device of the 18th, 19th or 20th aspect, the treatment unit and the return unit are connected to each other and stored in a case.

### Effect of the Invention

### (Stock Solution Concentrating Device)

According to the 1st aspect of the invention, the filter separates a stock solution such as pleural and ascitic fluid or blood plasma into a filtrate from which a cell component has been removed and a separate liquid containing a cell component, and the concentrator further concentrates the filtrate. Thus, a concentrated solution can be obtained having a high concentration of an active component and without the cell component. Additionally, the supply amount adjustment function of the stock solution supply unit adjusts the amount of the stock solution to be supplied to the filter. The efficiency with which the stock solution such as pleural and ascitic fluid or blood plasma can be treated is increased by treating the stock solution at a flow rate at which the filtration member is not susceptible to the deposition of cells or the like. The separate liquid is again supplied to the filter through the circulation flow path so that it is possible to recover the active component leaked together with the separate liquid. Further, it is possible to allow the circulation flow to always flow between the filter and the circulation flow path. This enables the cells to be hardly adhered to the filtration member. Therefore, it is possible to reduce a phenomenon such as the deposition of cells on the filtration member, and thus it is not necessary to clean the filtration member for a long period of time. Accordingly, it is possible to prevent a reduction in the treatment efficiency of the stock solution such as pleural and ascitic fluid or blood plasma.

According to the 2nd aspect of the invention, the filter separates a stock solution such as pleural and ascitic fluid or blood plasma into a filtrate from which a cell component has been removed and a separate liquid containing a cell component, and the concentrator further concentrates the filtrate. Thus, a concentrated solution can be obtained having a high concentration of an active component and without the cell component. Additionally, the supply amount adjustment function of the stock solution supply unit adjusts the amount of the stock solution to be supplied to the filter. The efficiency with which the stock solution such as pleural and ascitic fluid or blood plasma can be treated is increased by treating the stock solution at a flow rate at which the filtration member is not susceptible to the deposition of cells or the like. The separate liquid is again supplied to the filter through the circulation flow path so that it is possible to recover the active component leaked together with the separate liquid. Further, it is possible to allow the circulation flow to always flow between the filter and the circulation flow path. This enables the cells to be hardly adhered to the filtration member. Therefore, it is possible to reduce a phenomenon such as the deposition of cells on the filtration member, and thus it is not necessary to clean the filtration member for a long period of time. Accordingly, it is possible to prevent a reduction in the treatment efficiency of the stock solution such as pleural and ascitic fluid or blood plasma. The pleural and ascitic fluid supplied from the nozzle forms a circulation flow in the circulation flow path. As compared to the case of using a pump or the like to form a circulation flow, it is possible to reduce a stimulus to the stock solution or the cells circulated through the circulation flow path. Thus, it is possible to reduce the production of substances induced by inflammation such as cytokines, associated with the activation of the cell component which is caused when the stimulus is given to the cells and to reduce changes in cells (changes in surface markers) in reusing the collected cancer cells, lymphocytes, and macrophages. Further, the cancer cells, lymphocytes, and macrophages can be collected in a state close to the state of being present in the body. Thus, the collected cancer cells, lymphocytes, and macrophages can be also used for various therapies (e.g., production of cancer vaccines, selection of optimal anticancer drugs, selection of optimal anticancer drugs, and immunotherapy).

According to the 3rd aspect of the invention, since the flow generating means is disposed at the rear of the filter, the cells in the pleural and ascitic fluid are not in contact with the flow generating means. It is possible to reduce a stimulus applied to the cells in the pleural and ascitic fluid. Thus, it is possible to reduce the production of substances induced by inflammation such as cytokines, associated with the activation of the cell component which is caused when the stimulus is given to the cells and to reduce changes in cells (changes in surface markers) in reusing the collected cancer cells, lymphocytes, and macrophages.

According to the 4th aspect of the invention, the circulation flow in the circulation flow path can be made more smoothly, and the flow of the stock solution in the filter or the circulation flow path can be stabilized. Accordingly, it is possible to stabilize the filtered state of the stock solution.

According to the 5th aspect of the invention, the pressurization mechanism pressurizes the stock solution in the stock solution storage part and supplies the stock solution from the nozzle to the circulation flow path. As compared to the case of using a pump or the like to supply the stock solution in the stock solution storage part to the nozzle, it is possible to further reduce a stimulus to the cells contained in the stock solution.

According to the 6th aspect of the invention, the water removing means separates a predetermined amount of water from the filtrate. Thus, the pressurizing force pressurizing the stock solution is adjusted based on the flow rate of the concentrated solution discharged from the concentrator so that the concentration rate of the concentrated solution can be adjusted.

According to the 7th aspect of the invention, the concentrated solution discharge means discharges a predetermined amount of the concentrated solution from the concentrator. Thus, the pressurizing force pressurizing the stock solution is adjusted based on the flow rate of the water separated from the concentrated solution discharged from the concentrator so that the concentration rate of the concentrated solution can be adjusted.

According to the 8th aspect of the invention, the pressurizing force pressurizing the stock solution is adjusted based on the remaining amount of the stock solution and the flow rates of the concentrated solution and water discharged from the concentrator so that the concentration rate of the concentrated solution can be adjusted.

According to the 9th aspect of the invention, after the end of (or in the middle of) the treatment of the pleural and ascitic fluid, the liquid and/or residue in the circulation flow path can be discharged from the separated product recovery unit. Thus, when cancer cells, lymphocytes, and macrophages are contained in the pleural and ascitic fluid, these cells can be collected in a state close to the state of being present in the body. That is, the stock solution concentrating device, stock solution treatment device, and circulation-type treatment device of the present invention can be used as cell recovery devices. Further, the cells can be collected by supplying a liquid for cleaning the circulation circuit or the filter.

According to the 10th aspect of the invention, the concentrated solution storage containers are attached to the branch pipelines of the concentrated solution recovery pipe so that the concentrated solution can be sequentially stored in the concentrated solution storage containers. Thus, the concentrated solution storage containers storing a predetermined amount of the concentrated solution can be sequentially used for drip infusion. Additionally, during the use of one concentrated solution storage container for drip infusion, the concentrated solution can be stored in the other concentrated solution storage container. This enables the stock solution to be continuously treated and also enables the treatment to be performed at the bedside.

According to the 11th aspect of the invention, the concentrated solution storage containers for supplying the concentrated solution can be switched automatically. Additionally, it is not necessary to use a special instrument and perform a control process in order to switch the concentrated solution storage containers. This results in simplification of the structure of the device. Further, it is possible to suppress the occurrence of an error in recovery of the concentrated solution due to malfunction of the device. This results in improvement in handling properties of the device. According to the 12th aspect of the invention, the concentrated solution bags communicated with the concentrator are sequentially switched by a flow rate adjusting members, during the use of one concentrated solution bag for drip infusion, the concentrated solution can be stored in the other concentrated solution bag. Accordingly, the treatment of the stock solution can be performed in parallel to the administration of the concentrated solution. Consequently, it is possible to reduce the time required for the treatment of administering the concentrated water after the treatment to the patient while treating the pleural and ascitic fluid taken from the patient as the stock solution (cell-free and concentrated ascites reinfusion therapy).

According to the 13th aspect of the invention, the clogging of the filtration member can be suppressed.

### (Stock Solution Treatment Device)

According to the 14th aspect of the invention, the concentrated solution storage containers are attached to the branch pipelines of the concentrated solution recovery pipe so that the concentrated solution can be sequentially stored in the concentrated solution storage containers. Thus, the concentrated solution storage containers storing a predetermined amount of the concentrated solution can be sequentially used for drip infusion. Additionally, during the use of one concentrated solution storage container for drip infusion, the concentrated solution can be stored in the other concentrated solution storage container. This enables the stock solution to be continuously treated and also enables the treatment to be performed at the bedside.

According to the 15th aspect of the invention, the recovery of the pleural and ascitic fluid discharged from the patient can be performed simultaneously with the supply of the recovered pleural and ascitic fluid to the filter of the ascitic fluid concentration device. Additionally, the recovery of the pleural and ascitic fluid and the supply of the pleural and ascitic fluid to the filter can be continuously performed. Even if the recovery of the pleural and ascitic fluid and the supply of the pleural and ascitic fluid to the filter is continuously performed, there is no influence on the patient.

According to the 16th aspect of the invention, the stock solution can be sequentially recovered in the recovery chambers of the recovery sheet. This allows the stock solution to be continuously recovered. When the front space is pressurized, the stock solution is flowed into the rear space through the separation part, and the stock solution is discharged from the stock solution discharge port to the outside. Thus, the recovery chambers are sequentially pressurized so that the stock solution can be continuously discharged to the outside.

According to the 17th aspect of the invention, when the recovery sheet is wound by the winding unit, the recovery sheet is sequentially supplied to the pressurization unit. This allows the recovery chambers to be easily and sequentially pressurized.

### (Circulation Treatment Device)

According to the 18th aspect of the invention, the stock solution such as pleural and ascitic fluid or blood plasma is recovered from the living body, the extracorporeal treatment is performed, and the stock solution is returned to the living body. Thus, it is possible to reduce the treatment time, compared to the offline treatment. Further, the stock solution discharged from the living body is once stored in the reservoir. Therefore, even in the case where the treatment unit transports the stock solution via the pump or negative pressure, it is possible to prevent the influence of the negative pressure on the living body. Furthermore, it is possible to continuously treat the stock solution. Once the device is set up, the operator does not have to always monitor the device. This can reduce the burden of the operator. Additionally, it is possible to reduce the treatment time. This results in a significant reduction in the patient's burden.

According to the 19th aspect of the invention, the state of the stock solution flowing in the recovery tube is checked by a sensor. Thus, it is possible to operate the device so as not to apply negative pressure to the living body.

According to the 20th aspect of the invention, the treatment unit and the return unit are connected to each other and stored in a case. This makes preparations for the treatment easy. Additionally, the treatment can be performed by preparing the case. Consequently, it is possible to perform the whole treatment at the bedside in the patient's room and it is also possible to perform the treatment in the patient's house.

### BRIEF DESCRIPTION OF THE DRAWINGS

FIG. 1 is an outline explanatory view of a stock solution concentrating device 1A of an embodiment.

FIG. 2 is an outline explanatory view of a stock solution concentrating device 1B of another embodiment.

FIG. 3 is an outline explanatory view of a stock solution concentrating device 1C of another embodiment.

FIG. 4A is a view showing another shape of a nozzle 36, and FIG. 4B is a view showing another shape of a circulation flow path 30.

FIG. 5 is an outline explanatory view of a stock solution treatment device of an embodiment.

FIG. 6 is a view showing a time schedule for recovering pleural and ascitic fluid and returning it to the body via drip infusion.

FIGS. 7A and 7B are views showing an example of a recovery unit 50.

FIG. 8 is an outline explanatory view of a stock solution treatment device of another embodiment.

FIGS. 9A to 9C are views of other examples of the recovery unit 50.

### MODE FOR CARRYING OUT THE INVENTION

The stock solution concentrating device of the present invention is a device for obtaining a treatment solution by filtering and concentrating a stock solution such as pleural and ascitic fluid which can be administered to a patient by a method such as intravenous infusion or intraperitoneal injection, wherein the device can prevent clogging of the filtration member even in the case of continuously filtering the stock solution through the filtration member.

Further, the stock solution treatment device of the present invention is a device for which the stock solution concentrating device of the present invention is employed, wherein the stock solution can be continuously treated, and the solution (concentrated solution) treated by the stock solution concentrating device (filtration and concentration) can be continuously administered to a patient.

Further, the circulation treatment device of the present invention is a device capable of continuously recovering the pleural and ascitic fluid drained from the thoracic and peritoneal cavities of a patient and of directly returning the treatment solution after treatment to the patient. In other words, the circulation treatment device of the present invention is a device for extracorporeal circulation of the stock solution such as pleural and ascitic fluid.

There is no particular limitation as to the stock solution that is treated by the stock solution treatment device and the stock solution concentrating device of the present invention. For example, pleural and ascitic fluid or blood plasma can be used. Note that, in the circulation treatment device of the present invention, the pleural and ascitic fluid is used as the stock solution.

The pleural and ascitic fluid is pleural fluid or ascitic fluid which is accumulated in the thoracic and peritoneal cavities, caused by carcinomatous pleurisy and hepatic cirrhosis. The pleural and ascitic fluid contains plasma components leaked from the blood vessels or organs (e.g., proteins, hormones, sugars, lipids, electrolytes, vitamins, bilirubin, and amino acids), hemoglobin, cancer cells, lymphocytes, macrophages, histiocytes, leukocytes, erythrocytes, platelets, and bacteria. In the stock solution concentrating device of the present invention, it is possible to produce a concentrated solution containing water and blood plasma contained in the pleural and ascitic fluid and other useful components (e.g., proteins such as albumin and globulin, hereinafter referred to as useful components) by removing cancer cells, macrophages, histiocytes, leukocytes, erythrocytes, platelets, and bacteria from this pleural and ascitic fluid.

The blood plasma is, for example, plasma waste fluid discarded in plasma exchange. Hence, the plasma waste fluid is purified by the stock solution concentrating device and the stock solution treatment device of the present invention so that reusable regenerated blood plasma can be produced. Note that, in this case, in the stock solution concentrating device of the present invention, a plasma component separator may be used in place of the concentrator.

There is no particular limitation as to the filtration member used in the stock solution concentrating device of the present invention so long as the filtration member can permeate through the blood plasma, water, and useful components contained in the pleural and ascitic fluid, but does not permeate through cell components such as cancer cells, lymphocytes, macrophages, histiocytes, leukocytes, erythrocytes, platelets, and bacteria. Examples of filtration members include filtration members which are used as ascites filters for CART such as hollow fiber membranes, plasma separators for plasma exchange or plasma component separators for plasma exchange; and non-woven fabrics used for leukocytapheresis.

### (Stock Solution Concentrating Device 1A of Embodiment)

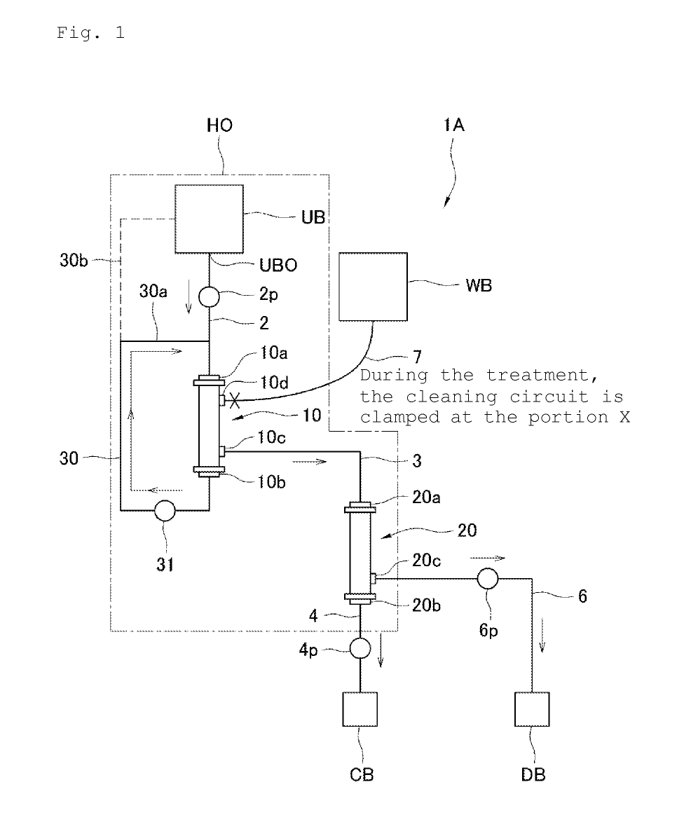

The stock solution concentrating device 1A of the embodiment will be described with reference to FIG. 1.

Hereinafter, the case where the stock solution to be treated is the pleural and ascitic fluid will be described as a representative example.

In FIG. 1, a symbol UB denotes a stock solution bag that stores a stock solution, i.e., pleural and ascitic fluid recovered from the thorax or abdomen. Further, a symbol CB denotes a concentrated solution bag that stores a concentrated solution obtained by filtering and concentrating the stock solution. Furthermore, a symbol DB denotes a waste solution bag that stores water separated from the filtrate.

### (Filter 10)

A symbol 10 denotes a filter that filters the pleural and ascitic fluid supplied from the stock solution bag UB. In other words, the filter 10 stores a filtration member, filters the pleural and ascitic fluid through the filtration member, and separates the fluid into a filtrate and a separate liquid containing cells. For example, an ascites filter for CART, a plasma separator used for plasma exchange or a plasma component separator can be used as the filter 10.

The filter 10 will be specifically described. The filter 10 comprises a supply port 10a that is communicated with a liquid discharge port UBO of the stock solution bag UB through a tube 2. In other words, a liquid to be filtered, i.e., a stock solution is supplied from the supply port 10a to the filter 10.

Further, the filter 10 comprises a filtrate discharge port 10c that is separated from the supply port 10a by the filtration member and a separate liquid discharge port 10b that is communicated with the supply port 10a through a space between filtration members.

The filter 10 comprises a filtration member. As described above, this filtration member has a function that permeates through water, blood plasma, and useful components such as useful proteins, but does not permeate through cell components such as cancer cells, macrophages, histiocytes, leukocytes, erythrocytes, platelets, and bacteria.

For this reason, when the stock solution is supplied from the supply port 10a to the filter 10, the stock solution is filtered through the filtration member, and the filtrate containing useful components is discharged from the filtrate discharge port 10c. On the other hand, when the cell components, the useful components, the blood plasma, and even the water are not passed through the filtration member, each of the components is discharged as a separate liquid from the separate liquid discharge port 10b.

Note that the separate liquid discharged from the separate liquid discharge port 10b is again returned to the supply port 10a or the tube 2 through the circulation flow path 30. The details will be described later.

As the system for supplying the pleural and ascitic fluid from the stock solution bag UB to the filter 10, both of the drop system and the pump system can be employed. In the case of the drop system, the stock solution bag UB itself corresponds to one having the supply amount adjustment function of the stock solution supply unit, which is described in the claims. In the case of the pump system, the pump itself corresponds to one having the supply amount adjustment function of the stock solution supply unit, which is described in the claims. In these cases, as one having the supply amount adjustment function, it is preferable to provide on the tube 2 with a flow rate adjusting means 2p that adjusts the flow rate of the pleural and ascitic fluid. The flow rate adjusting means 2p is not particularly limited. In the case of the pump system, a flow rate-adjustable pump can be used as the flow rate adjusting means 2p. In the case of the drop system, a clamp (a fastening tool for forming a constriction in the flow path) attached to the tube 2 can be used as the flow rate adjusting means 2p.

Note that, in the case of the drop system, even if a clamp (refer to 4p or 6p of FIG. 1) is provided on one or both of a tube 4 that connects the concentrator 20 to the concentrated solution bag CB or a tube 6 that connects the concentrator 20 to the waste solution bag DB instead of providing the clamp on the tube 2, it is possible to adjust a flow rate for supplying the pleural and ascitic fluid from the stock solution bag UB to the filter 10.

Further, in the case of employing the pump system, a pump (corresponding to the flow generating means, which is described in the claims) may be provided on a tube 3 that connects the filtrate discharge port 10c of the filter 10 to the concentrator 20. In this case, when the pump is operated, negative pressure is generated in the tube 3. The negative pressure can generate a flow of the stock solution flowing from the filter 10 to the tube 3. Thus, it is possible to make the inside of the filter 10 negative in pressure. Thus, the negative pressure can generate a flow of the stock solution flowing from the stock solution bag UB to the filter 10. In the case of this system, the pump is in contact with only the filtrate from which cells in the ascitic fluid before filtration have been removed. Hence, the cells in the pleural and ascitic fluid are not in contact with the pump so that it is possible to reduce a stimulus to the cells in the pleural and ascitic fluid. Thus, it is possible to reduce the production of substances induced by inflammation such as cytokines, associated with the activation of the cell component which is caused when the stimulus is given to the cells and to reduce changes in cells (changes in surface markers) in reusing the collected cancer cells, lymphocytes, and macrophages.

### (Concentrator 20)

A symbol 20 denotes a concentrator that concentrates the filtrate supplied from the filter 10. The concentrator 20 has a structure substantially similar to that of the filter 10 and has a function that separates water from the filtrate to form a concentrated solution. In other words, the concentrator 20 has a structure in which a water separating member having a function that separates water from the filtrate is stored in place of a separating member of the filter 10. For example, an ascitic fluid concentrator which is used for CART or a dialysis filter which is used for dialysis can be used as the concentrator 20.

The concentrator 20 will be specifically described. The concentrator 20 includes a supply port 20a that is communicated with the filtrate discharge port 10c of the filter 10 through the tube 3. In other words, the liquid to be concentrated, i.e., the filtrate, is supplied from the supply port 20a to the concentrator 20.

Further, the concentrator 20 includes a water discharge port 20c that discharges the liquid separated from the filtrate, i.e., water and a concentrated solution discharge port 20b that discharges the liquid concentrated by removal of water, i.e., a concentrated solution.

The concentrator 20 includes a water separating member. The water separating member has a function that permeates through water, but does not permeate through useful components such as useful proteins contained in the blood plasma.

For this reason, when the pleural and ascitic fluid is supplied from the supply port 20a to the concentrator 20, water is separated from the filtrate through the water separating member, the separated water is discharged from the water discharge port 20c, and the water is supplied to the waste solution bag DB through the tube 6. On the other hand, the concentrated solution concentrated by removal of a part of water is discharged from the concentrated solution discharge port 20b, and the discharged concentrated solution is supplied to the concentrated solution bag CB through the tube 4.

Note that the tube 6 connected to the water discharge port 20c comprises a flow rate adjusting means 6p that adjusts the amount of water discharged from the water discharge port 20c. The amount (discharge flow rate) of water to be discharged is adjusted by the flow rate adjusting means 6p so that a rate to concentrate the filtrate (i.e., the concentration of the active component in the concentrated solution) can be adjusted. The flow rate adjusting means 6p is not particularly limited. A flow rate-adjustable pump can be used as the flow rate adjusting means 6p, and a clamp or the like can be used as the flow rate adjusting means 6p.

In the case of employing the pump as the flow rate adjusting means 6p, the tube 6 between the water discharge port 20c and the flow rate adjusting means 6p becomes negative in pressure with respect to the concentrator 20. Hence, the pump produces an effect of sucking water in the concentrator 20 from the water discharge port 20c. In the case of this configuration, when the operation of the pump is controlled to an extent that the negative pressure generated by the operation of the pump does not affect on the filter 10, it is possible to prevent the filtered state in the filter 10 from being affected by the negative pressure.

Further, a flow rate adjusting means 4p such as a pump or clamp may be provided on the tube 4, in place of the tube 6. The flow rate adjusting means 4p and 6p may be provided on both the tubes 6 and 4. In the case of providing the flow rate adjusting means 4p and 6p on both the tubes 6 and 4, the flow rate and discharge flow rate of the concentrated solution discharged from the concentrator 20 are adjusted so that the rate concentrated by the concentrator 20 can be accurately controlled. In the case of keeping the discharge flow rate constant, the flow rate of the concentrated solution is just adjusted by the flow rate adjusting means 4p so that the rate concentrated by the concentrator 20 can be controlled.

Note that, in the case of providing the flow rate adjusting means 4p and 6p on both the tubes 6 and 4, the same device (pump or clamp) or different devices may be used for both the flow rate adjusting means 4p and 6p. For example, a pump or clamp may be used for both the flow rate adjusting means 4p and 6p. Alternatively, a pump (or clamp) may be used for the flow rate adjusting means 4p, and a clamp (or pump) may be used for the flow rate adjusting means 6p.

In the case of employing the pump as the flow rate adjusting means 4p, the operation of the pump is controlled, similarly to the case of employing the pump as the flow rate adjusting means 6p. In other words, the operation of the pump is controlled to an extent that the negative pressure generated by the operation of the pump does not affect on the filter 10. Thus, even if the pump is employed as the flow rate adjusting means 4p, it is possible to prevent the filtered state in the filter 10 from being affected.

### (Circulation Flow Path 30)

As shown in FIG. 1, the stock solution concentrating device 1A of the embodiment includes a circulation flow path 30 that returns the separate liquid discharged from the separate liquid discharge port 10b of the filter 10 to the supply port 10a or the tube 2. The circulation flow path 30 is a pipeline such as a tube, and is equipped with a circulation flow forming means 31. The circulation flow forming means 31 forms a flow of the separate liquid flowing from the separate liquid discharge port 10b to the supply port 10a in the circulation flow path 30. In other words, the circulation flow forming means 31 can form a circulation flow circulating between the filter 10 and the circulation flow path 30. The circulation flow forming means 31 is not particularly limited. Examples thereof include general pumps such as roller pumps, axial flow pumps, and centrifugal pumps.

The circulation flow path 30 is provided and the above flow is formed by the circulation flow forming means 31 so that the stock solution (pleural and ascitic fluid) supplied from the stock solution bag UB through the tube 2 as well as the separate liquid discharged from the separate liquid discharge port 10b can be supplied to the filter 10.

Thus, the active component leaked together with the separate liquid is passed many times through the filter 10 so that it is possible to improve the recovery efficiency of the active component contained in the pleural and ascitic fluid.

Not only the flow passing through the filtration member but also the flow of the liquid (pleural and ascitic fluid and separate liquid) along the filtration member are formed in the filter 10 so that the surface of the filtration member can be made in a state close to the state of always being cleaned with the liquid. Thus, it is possible to prevent the filtration member from being clogged by the cells adhered to the surface of the filtration member.

When the clogging of the filtration member can be prevented, it is not necessary to clean the filtration member for a long period of time. Accordingly, it is possible to prevent a reduction in the treatment efficiency of the pleural and ascitic fluid.

In the case where, particularly, a hollow fiber membrane is used as the filtration member and the pleural and ascitic fluid is allowed to flow into the hollow fiber membrane, the cells contained in the pleural and ascitic fluid are easily passed through the center of the hollow fiber membrane. This allows the cells to be hardly adhered to the inner surface of the hollow fiber membrane. Accordingly, when the hollow fiber membrane is used as the filtration member, it is possible to reduce the possibility of clogging of the filtration member due to the cells adhered to the surface of the filtration member.

### (Heating Mechanism)

As described above, the clogging of the filtration member can be prevented by providing the circulation flow path 30. In order to prevent the clogging of the filtration member, it is preferable to provide the circulation flow path 30 and to provide a mechanism for heating the stock solution (and/or filtrate). When the stock solution (and/or filtrate) is maintained at a temperature of from about 28° C. to 42° C., preferably at a temperature of from about 36° C. to 38° C., and more preferably at a temperature of about 37° C., the clogging of the filtration member can be suppressed.

For example, when a thermostatic container HO capable of storing the whole of the filter 10, the concentrator 20, and the circulation flow path 30 is provided (refer to FIG. 1), and the thermostatic container HO stores the filter 10, the concentrator 20, and the circulation flow path 30 as well as the tubes 2 and 3 which connect these parts, it is possible to maintain the stock solution (and/or filtrate) at a temperature of from about 28° C. to 42° C., preferably at a temperature of from about 36° C. to 38° C., and more preferably at a temperature of about 37° C.

Further, the above temperature may be maintained by covering the filter 10, the concentrator 20, the circulation flow path 30, and the tubes 2 and 3 with a thermal insulation member having a thermal insulation function, providing a heating device such as a heater, and heating the stock solution (and/or filtrate). (Plurality of Concentrated Solution Bags CB)

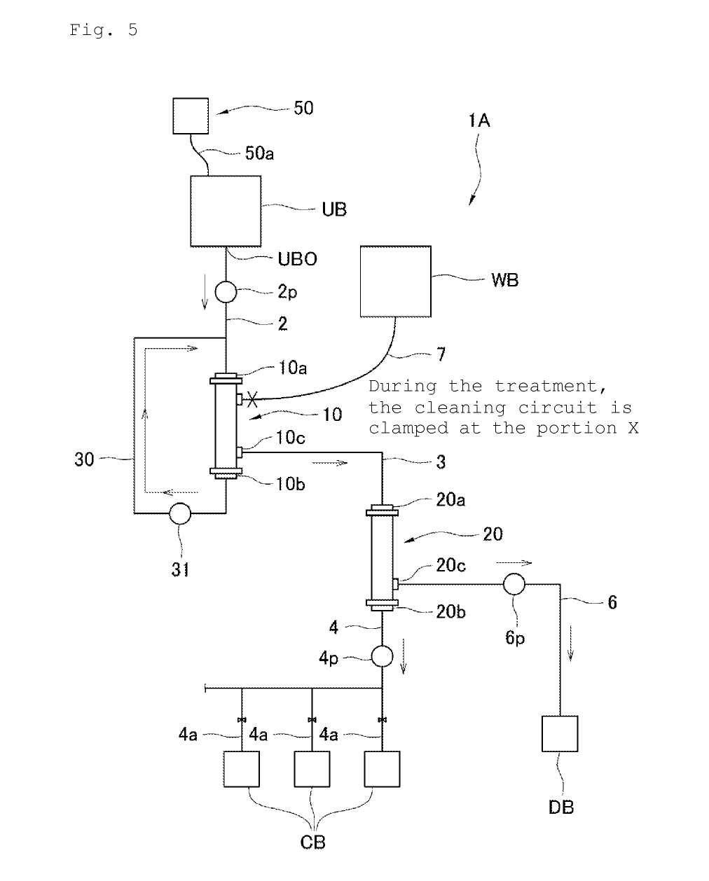

Note that in the stock solution concentrating device 1A, the tube 4 that discharges the concentrated solution from the concentrator 20 (corresponding to the concentrated solution recovery pipe, which is described in the claims) includes a plurality of branch pipelines 4a. The concentrated solution bags CB may be detachably attached to each of the branch pipelines 4a (refer to FIG. 5). In the case of this configuration, a flow rate adjusting member that stops the flow of the solution in each of the branch pipelines 4a or changes the flow rate, such as a clamp, is provided at the branch pipelines 4a. In other words, the flow rate adjusting member is configured to be capable of establishing or blocking the communication between each of the concentrated solution bags CB and the concentrator 20.

With this configuration, the concentrated solution bags CB communicated with the concentrator 20 are sequentially switched, whereby it is possible to adjust such that when a predetermined amount of the concentrated solution is stored in one concentrated solution bag CB, the concentrated solution is supplied to the other concentrated solution bag CB. Thus, during the use of one concentrated solution bag CB for drip infusion, the concentrated solution can be stored in the other concentrated solution bag CB. Accordingly, the concentrated solution can be administered to a patient while continuously performing the treatment of the stock solution. In other words, the treatment of the stock solution can be performed in parallel to the administration of the concentrated solution. Consequently, it is possible to reduce the time required for the treatment of administering the concentrated water after the treatment to the patient while treating the pleural and ascitic fluid taken from the patient as the stock solution (cell-free and concentrated ascites reinfusion therapy).

Particularly, it is preferable to provide the concentrated solution bags CB so as to form a difference in height of each of the concentrated solution bags CB. With this configuration, the concentrated solution bags CB for supplying the concentrated solution can be switched automatically. Additionally, it is not necessary to use a special instrument and perform a control process in order to switch the concentrated solution bags CB. This results in simplification of the structure of the device. Further, it is possible to suppress the occurrence of an error in recovery of the concentrated solution due to malfunction of the device. This results in improvement in handling properties of the device.

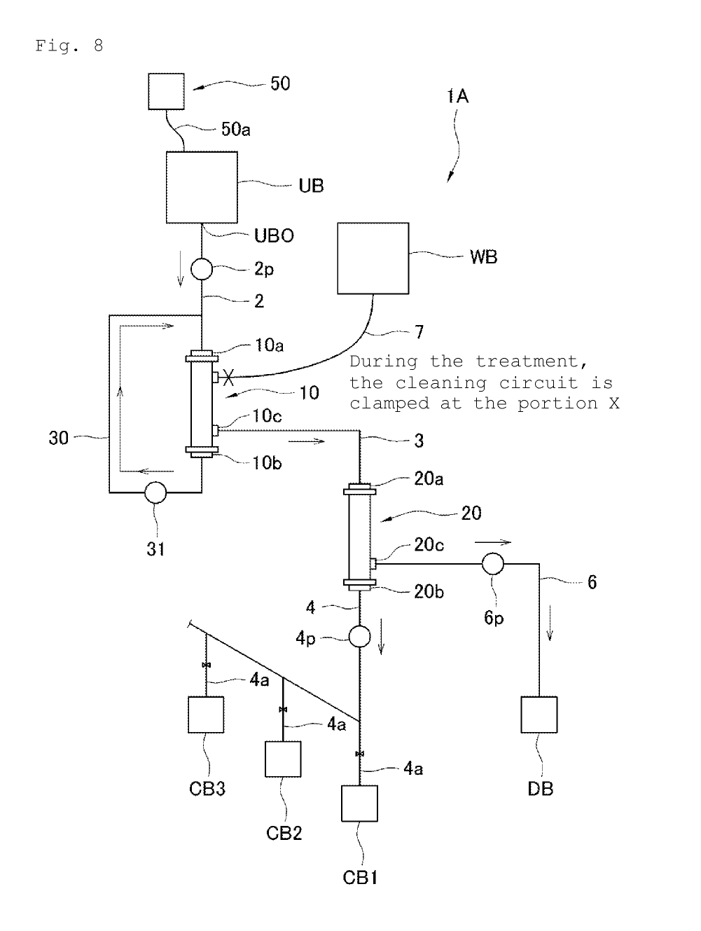

For example, as shown in FIG. 8, the concentrated solution bags CB are disposed so that the height of each position (specifically each position connected to each of the branch pipelines 4a) increases in the order of a first concentrated solution bag CB1, a second concentrated solution bag CB2, and a third concentrated solution bag CB3. Thus, the concentrated water is supplied only to the first concentrated solution bag CB1 until the first concentrated solution bag CB1 is filled up. The concentrated solution is not supplied to the other concentrated solution bag CB. On the other hand, when the first concentrated solution bag CB1 is filled up, the concentrated solution is supplied to the second concentrated solution bag CB2 through the branch pipeline 4a. In this state, the concentrated solution bag CB1 can be detached from the branch pipeline 4a by blocking the flow path between the first concentrated solution bag CB1 and the branch pipeline 4a by a clamp or the like. Accordingly, it becomes possible to administer the concentrated solution in the concentrated solution bag CB1 to the patient.

Further, even if the concentrated solution bag CB1 is detached from the branch pipeline 4a, the concentrated solution can be continuously supplied to the second concentrated solution bag CB2. Thus, it is possible to continuously perform the treatment of the stock solution.

Then, when the second concentrated solution bag CB2 is filled up, the concentrated solution is supplied to the third concentrated solution bag CB3 through the branch pipeline 4a. In other words, when a concentrated solution bag CB at a lower position is filled up, the concentrated solution is automatically supplied to a concentrated solution bag CB at a higher position.

According to the configuration, the concentrated solution bags CB1 to CB3 for supplying the concentrated solution can be switched automatically. Additionally, it is not necessary to use a special instrument and perform a control process in order to switch the concentrated solution bags CB. This results in simplification of the structure of the device. Further, it is possible to suppress the occurrence of an error in recovery of the concentrated solution due to malfunction of the device. This results in improvement in handling properties of the device.

### (Another Circulation Flow Path 30)

The above example has described the case where the circulation flow path 30 is provided so as to communicate with the filter 10, the separate liquid discharge port 10b, and the supply port 10a (or the tube 2). The circulation flow path 30 may be provided so as to communicate the separate liquid discharge port 10b with the stock solution bag UB. In other words, it may be configured to return the stock solution discharged from the separate liquid discharge port 10b to the stock solution bag UB. In this case, the circulation flow in the circulation flow path 30 can be made more smoothly. In the case of the above example, the two flows join together in the supply port 10a (or the tube 2), and thus a confluent part of the flows may be disturbed. However, when the stock solution is returned to the stock solution bag UB, the problem does not occur. Thus, it is possible to stabilize the flow of the stock solution in the filter 10 or the circulation flow path 30. Accordingly, the filtered state of the stock solution can be stabilized. Specifically, as shown in FIG. 1, a flow path 30b that is communicated with the stock solution bag UB is provided in place of a flow path 30a connected to the supply port 10a (or the tube 2), so that it is possible to form the circulation flow path 30 that returns the stock solution to the stock solution bag UB.

### (Stock Solution Concentrating Device 1B of Another Embodiment)

In the stock solution concentrating device 1A of the embodiment, the described case is that the circulation flow path 30 is provided separately from the tube 2 that supplies the pleural and ascitic fluid from the stock solution bag UB to the filter 10. However, the circulation flow path 30 itself may be a flow path that supplies the pleural and ascitic fluid from the stock solution bag UB to the filter 10.

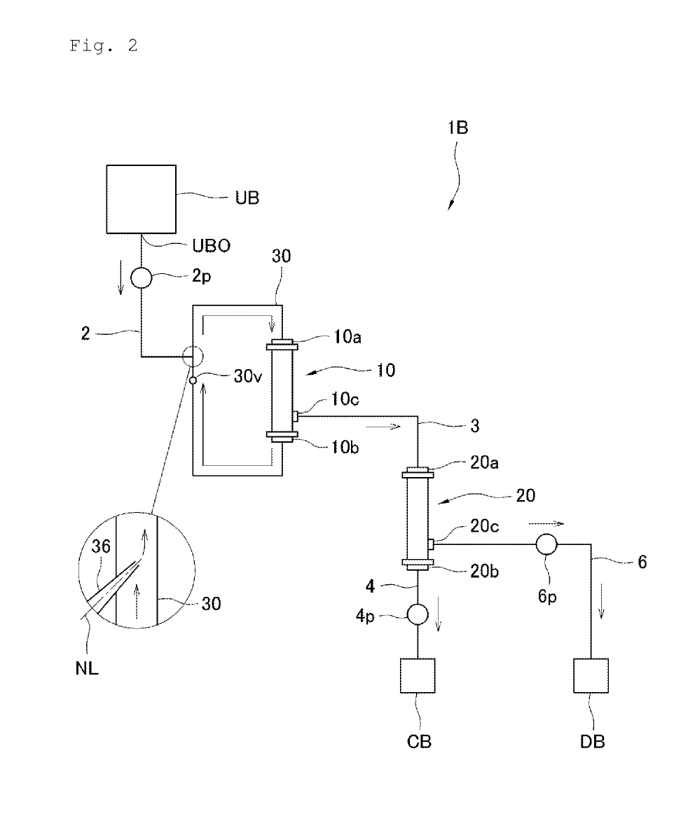

Hereinafter, the stock solution concentrating device 1B in which the circulation flow path 30 itself is a flow path for supplying the pleural and ascitic fluid from the stock solution bag UB to the filter 10 will be described with reference to FIG. 2.

Note that, the stock solution concentrating device 1B has a structural function substantially similar to that of the stock solution concentrating device 1A except that the circulation flow path 30 itself is a flow path for supplying the pleural and ascitic fluid from the stock solution bag UB to the filter 10, and the description therefor will be omitted, if appropriate.

As shown in FIG. 2, in the stock solution concentrating device 1B, only the circulation flow path 30 is connected to the supply port 10a of the filter 10. As described above, the circulation flow path 30 is a pipeline such as a tube. One end of the path is connected to the separate liquid discharge port 10b of the filter 10, and the other end is connected to the supply port 10a of the filter 10.

### (Raw Material Supply Means)

As shown in FIG. 2, a nozzle 36 of a pleural and ascitic fluid supplier that supplies the pleural and ascitic fluid from the stock solution bag UB to the filter 10 is provided in the middle of the circulation flow path 30 in the stock solution concentrating device 1B.

The end of the nozzle 36 is connected to the circulation flow path 30, and is communicated with the circulation flow path 30 through an opening at the end of the nozzle. Note that FIG. 2 shows that the end of the nozzle 36 is disposed in the circulation flow path 30.

The base end of the nozzle 36 is communicated with the liquid discharge port UBO of the stock solution bag UB via the tube 2. In other words, when the pleural and ascitic fluid is supplied from the stock solution bag UB to the nozzle 36, the pleural and ascitic fluid is supplied from the end of the nozzle 36 to the circulation flow path 30.

Further, the nozzle 36 is disposed such that the end opening faces the direction of the supply port 10a. In other words, the nozzle 36 is disposed such that the pleural and ascitic fluid, which has been supplied from the end opening of the nozzle 36 to the circulation flow path 30, flows in a direction from the separate liquid discharge port 10b of the filter 10 to the supply port 10a of the filter 10.

Thus, the water flow of the pleural and ascitic fluid, which has been supplied from the end opening of the nozzle 36 to the circulation flow path 30, can result in the formation of a flow flowing from the separate liquid discharge port 10b of the filter 10 to the supply port 10a of the filter 10 in the circulation flow path 30. Hence, the formation of the circulation flow flowing between the filter 10 and the circulation flow path 30 enables the nozzle 36 of the pleural and ascitic fluid supplier to serve as the circulation flow forming means 31 described above.

In this case, it is possible to reduce a stimulus to the cells circulating in the pleural and ascitic fluid or the circulation flow path 30, as compared to the case of using a pump as the circulation flow forming means 31. Thus, it is possible to reduce the production of substances induced by inflammation such as cytokines, associated with the activation of the cell component which is caused when the stimulus is given to the cells and to reduce influences on cells (changes in surface markers) in reusing the collected cancer cells, lymphocytes, and macrophages.

In the case where cells to be recovered are remained in the circulation flow path 30, it is possible to recover the cells which are less stimulated by the pump. Accordingly, not only the concentrated solution but also the cells remained in the circulation flow path 30 can be effectively used. That is, the stock solution concentrating device 1 of the embodiment can be used as a cell recovery device.

For example, after the end of (or in the middle of) the treatment of the pleural and ascitic fluid, the cells are recovered together with the liquid in the circulation flow path 30 so that the cancer cells, lymphocytes, and macrophages contained in the pleural and ascitic fluid can be collected in a state close to the state of being present in the body. Thus, the collected cancer cells can be used for various therapies (e.g., production of cancer vaccines, selection of optimal anticancer drugs, selection of optimal anticancer drugs, and immunotherapy).

The method for recovering cells is not particularly limited. In the case where a branch path is formed in the middle of the circulation flow path 30 and a valve is provided at the branch path, opening the valve enables the liquid in the circulation flow path 30 and the cells to be recovered. Further, in the case where the cells are recovered after the end of the treatment of the stock solution, physiological saline or the like is supplied from the tube 2 and then the cells are recovered together with physiological saline. In this case, a supply port for supplying physiological saline is provided at respective ports of the circulation flow path 30, the tube 3, and the filter 10 (for example, in the case where the filter 10 has a pressure monitor port 10d that detects internal pressure, the inlets are the pressure monitor ports 10d). Then, physiological saline is supplied from a cleaning solution bag WB so that it is possible supply physiological saline, for example, without detaching the stock solution bag UB after the end of the treatment. This makes the operation of recovering the cells easy.

Note that, in the case of the above configuration, the branch path or the valve provided at the branch path corresponds to the separated product recovery unit, which is described in the claims.

Further, it is not necessary to provide a special structure as the separated product recovery unit for recovering cells. For example, one end or the other end of the circulation flow path 30 is detached from the separate liquid discharge port 10b or the supply port 10a of the filter 10. Then, the cells can be recovered from the end thereof. In this case, one end or the other end of the circulation flow path 30 corresponds to the separated product recovery unit.

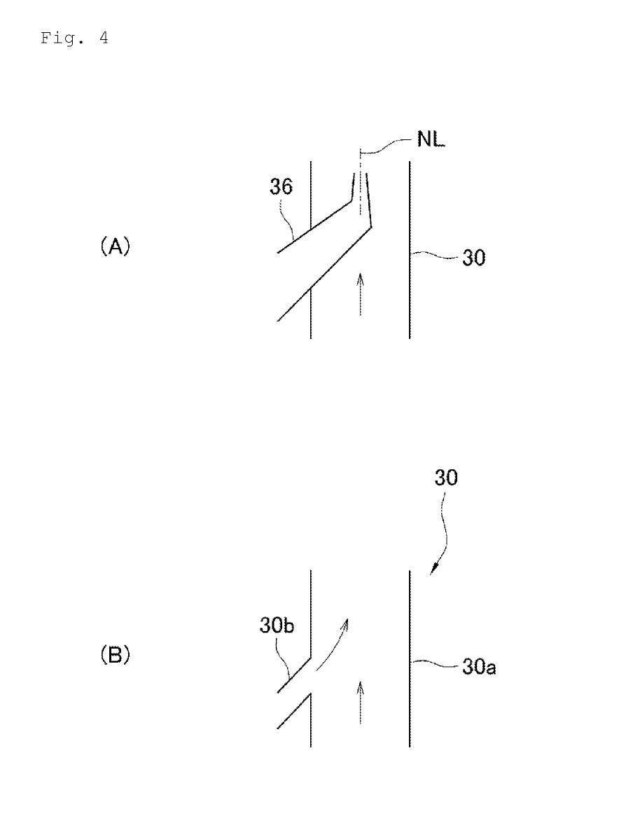

Note that the disposition and shape of the nozzle 36 is not particularly limited. The nozzle 36 may be disposed such that the stock solution supplied from the nozzle 36 to the circulation flow path 30 is a flow having a speed component in a direction from the separate liquid discharge port 10b of the filter 10 to the supply port 10a of the filter 10. In other words, the disposition and shape of the nozzle 36 may be configured such that when the stock solution is supplied from the nozzle 36 to the circulation flow path 30, the circulation flow described above is formed in the circulation flow path 30. In particular, as shown in FIG. 4 (A), when the nozzle is disposed such that an axis direction NL of the end of the nozzle 36 is parallel to the axis direction of the circulation flow path 30, the circulation flow described above can be reliably formed.

Further, when the circulation flow described above can be formed, the end of the nozzle 36 is not necessarily located in the circulation flow path 30. For example, as shown in FIG. 4 (B), a main flow path 30a and a joined flow path 30b inclined toward the axis direction of the main flow path 30a are provided in the circulation flow path 30. The joined flow path 30b may serve as the nozzle 36 described above.

Further, even if the nozzle 36 and the joined flow path 30b are not provided, the formation of a check valve 30v in the circulation flow path 30 enables the stock solution to flow in only one direction of the circulation flow path 30.

In the case of not forming a structure such as the joined flow path 30b, supplying the stock solution from the stock solution bag UB to the circulation flow path 30 through the tube 2 enables the stock solution to flow in the circulation flow path 30. However, it is not possible to control the direction of the flow of the stock solution. However, when the check valve 30v is provided in the middle of the circulation flow path 30, the stock solution can flow in only one direction. Hence, the check valve 30v is provided in the middle of the circulation flow path 30 so that it is possible to control the flow of the stock solution in the circulation flow path 30.

The flow of the stock solution in the circulation flow path 30 may be controlled by providing only the check valve 30v. However, when the joined flow path 30b and the nozzle 36 are provided and the check valve 30v is provided in the circulation flow path 30, it is possible to more reliably control the flow of the stock solution in the circulation flow path 30.

Note that the position of the check valve 30v to be provided is not particularly limited. However, the valve is preferably provided in a vicinity of a connection between the circulation flow path 30 and the tube 2, i.e., at the upstream side of the check valve 30v (at the side of the separate liquid discharge port 10b of the filter 10) (refer to FIG. 2).

The stock solution bag UB, the tube 2, or the nozzle 36 (or the joined flow path 30b) corresponds to the circulation flow forming means, which is described in the claims. In the case where the check valve 30v is provided on the circulation flow path 30, the check valve 30v constitutes the circulation flow forming means.

In the stock solution concentrating device 1B, the stock solution bag UB corresponds to the stock solution storage part, which is described in the claims.

Further, the stock solution bag UB or the tube 2 corresponds to the stock solution supply means, which is described in the claims.

### (Stock Solution Concentrating Device 1C of Another Embodiment)

As shown in FIG. 2, as for the stock solution concentrating device 1B, the described case is that the gravity or pump system for the nozzle 36 is employed as the system of supplying the stock solution from the stock solution bag UB.

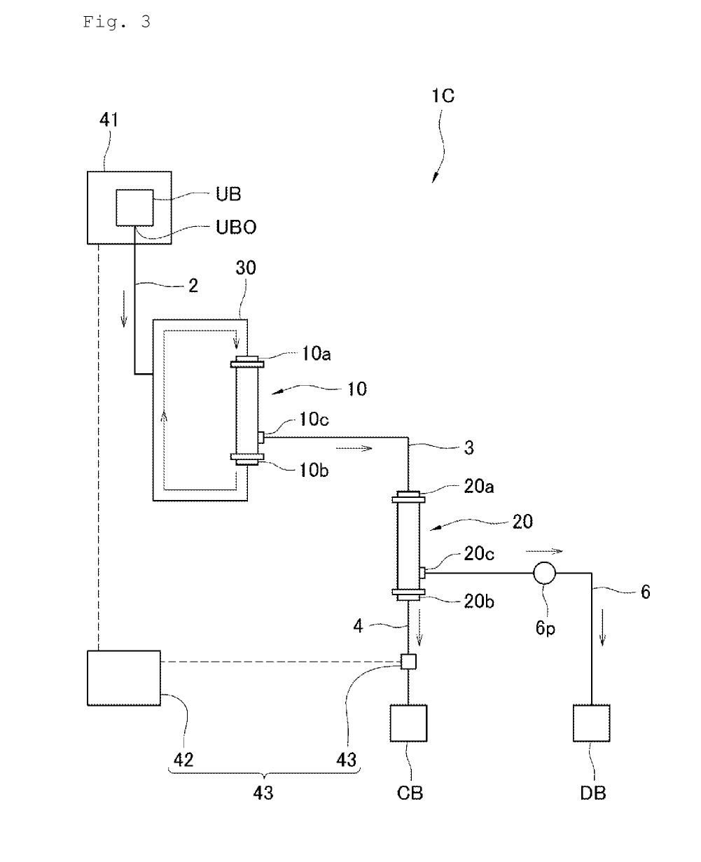

However, as shown in FIG. 3, a pressurization mechanism 40 comprising a pressurization means 41 that directly pressurizes the stock solution bag UB itself or the stock solution in the stock solution bag UB may be provided so that the stock solution is supplied to the nozzle 36 by the pressure generated by the pressurization means 41 of the pressurization mechanism 40. In this case, the obtained advantage is that a direct stimulus on the cells contained in the stock solution (for example, friction with the tube during passing through the tube) can be further reduced. Hence, the cells contained in the stock solution can be collected in a state close to the state of being present in the body, as compared to the case of using the stock solution concentrating device 1B.

There is no particular limitation as to the method for pressurizing the stock solution bag UB or the stock solution in the stock solution bag UB by the pressurization means 41. For example, a device capable of holding the stock solution bag UB and applying pressure may be employed as the pressurization means 41. Further, the stock solution bag UB is placed on a table, a weight or the like is placed thereon, and then pressure may be applied thereto. In this case, the weight corresponds to the pressurization means 41. The stock solution bag UB, i.e., the stock solution can be pressurized at a nearly constant force.

Further, it is possible to employ a method comprising the steps of: directly supplying gas (nitrogen, etc.), which has been pressurized from a cylinder or an air pump, to the stock solution bag UB or a closed container including the stock solution bag UB; and pressurizing the stock solution. In this case, the cylinder or the air pump corresponds to the pressurization means 41.

Further, in the case where the stock solution is supplied to the nozzle 36 by the pressure generated by the pressurization means 41 of the pressurization mechanism 40, the supply flow rate of the stock solution is indirectly adjusted by the force pressurizing the stock solution. For this reason, it is necessary to provide a pressurizing force controller 42 that adjusts the supply flow rate by adjusting the force pressurizing the stock solution. There is no particular limitation as to the mechanism or method for adjusting the supply flow rate by the pressurizing force controller 42. For example, the following method can be employed.

As shown in FIG. 3, a pump is provided on the tube 6 connected to the water discharge port 20c of the concentrator 20 as the flow rate adjusting means 6p. A flow rate detector 43 that measures the flow rate of the concentrated solution discharged from the concentrator 20 is provided on the tube 4.

In the case of the above configuration, the operation of the flow rate adjusting means 6p under constant conditions enables the flow rate (discharge flow rate) of water separated from the filtrate to be nearly constant, regardless of the flow rate of the filtrate supplied to the concentrator 20, in other words, the supply flow rate. Thus, the flow rate of the filtrate is the total of the discharge flow rate and the flow rate of the concentrated solution. Accordingly, the flow rate of the concentrated solution is measured by the flow rate detector 43 so that it is possible to grasp the supply flow rate.

Therefore, the pressurizing force controller 42 adjusts the operation of the pressurization means 41 based on the flow rate of the concentrated solution measured by the flow rate detector 43 so that the supply flow rate can be adjusted. As described above, the discharge flow rate is kept almost constant. Thus, the concentration rate of the filtrate and the concentration of the concentrated solution can be adjusted by simply changing the supply flow rate. Therefore, a solution having a desired concentration can be easily and stably obtained.

Note that the above example has described the case where the flow rate adjusting means 6p is operated under constant conditions. In the case where the pressurization means 41 is configured to pressurize the stock solution bag UB, i.e., the stock solution at a nearly constant force, the supply flow rate can be adjusted by controlling the operation of the flow rate adjusting means 6p. Of course, needless to say, the supply flow rate may be adjusted by controlling both the flow rate adjusting means 6p and the pressurization means 41.

In the stock solution concentrating device 1C, the flow rate adjusting means 6p corresponds to the water removing means, which is described in the claims.

### (Another Example of Stock Solution Concentrating Device 1C)

In the stock solution concentrating device 1C, the flow rate adjusting means 4p such as a roller pump may be provided on the tube 4 in place of the flow rate detector 43 (refer to FIGS. 1 and 2). In this case, even when the force pressurizing the stock solution is adjusted to a constant level by the pressurization means 41, the concentration rate of the concentrated solution can be adjusted by adjusting the flow rate of the concentrated solution discharged from the concentrator 20 by the flow rate adjusting means 4p. In other words, even if the force pressurizing the stock solution is not controlled with high accuracy by the pressurization means 41, the concentration rate of the concentrated solution can be adjusted with high accuracy. This results in an advantage of easily controlling the device.

### (Another Example of Stock Solution Concentrating Device 1C)

In the stock solution concentrating device 1C, a flow rate detector is provided on the tube 6, meanwhile, the flow rate adjusting means 4p such as a roller pump may be provided on the tube 4 in place of the flow rate detector 43. In this case, the flow rate of the concentrated solution discharged from the concentrator 20 is adjusted to a constant level by the flow rate adjusting means 4p. Thus, the pressurizing force controller 42 adjusts the operation of the pressurization means 41 based on the discharge flow rate of the concentrated solution measured by the flow rate detector so that the supply flow rate can be adjusted. Then, when the flow rate of the concentrated solution is kept almost constant, the concentration rate of the filtrate can be adjusted by simply changing the supply flow rate. Thus, the concentration of the concentrated solution can be adjusted. Therefore, a concentrated solution having a desired concentration can be easily and stably obtained.

Additionally, in the case of producing the concentrated solution used for CART, the discharge flow rate is usually larger than the flow rate of the concentrated solution. For example, since the concentration rate of the concentrated solution is usually about 10 times that of the filtrate, the discharge flow rate is about 9 times the flow rate of the concentrated solution. Thus, it is more accurate to measure the discharge flow rate than to measure the flow rate of the concentrated solution. Consequently, when the discharge flow rate is measured and the operation of the pressurization means 41 is adjusted based on the measured value, the supply flow rate can be controlled with high accuracy.

Note that in the case where the pressurization means 41 is configured to pressurize the stock solution bag UB, i.e., the stock solution at a nearly constant force, the supply flow rate can be adjusted by controlling the operation of the flow rate adjusting means 4p. Of course, needless to say, the supply flow rate may be adjusted by controlling both the flow rate adjusting means 4p and the pressurizing means 41.

As for the stock solution concentrating device 1C, in the case where a pump is provided on the tube 4 as the flow rate adjusting means 4p, similarly to the case of the flow rate adjusting means 6p, the operation of the flow rate adjusting means 4p is controlled to an extent that does not affect the filter 10 by the influence of the negative pressure caused by the flow rate adjusting means 4p provided on the tube 4. Thus, even if a pump is provided on the tube 4 as the flow rate adjusting means 4p, it is possible to prevent the filtered state in the filter 10 from being affected by the negative pressure caused by the flow rate adjusting means 4p.

### (Another Example of Stock Solution Concentrating Device 1C)

In the stock solution concentrating device 1C, the concentration rate of the concentrated solution is adjusted by directly measuring the flow rate. On the basis of the weight change of each of the solutions, the pressurizing force controller 42 may adjust the operation of the pressurization means 41.

For example, a function for measuring the weight of the stock solution bag UB, i.e., the weight of the stock solution is provided on the pressurization means 41. Further, there is provided a concentrated solution weight measurement unit and a water weight measurement unit, each of which measures the weight of the concentrated solution bag CB or the waste solution bag DB (i.e., the weight of the concentrated solution or water). Then, the measured weight is set to send to the pressurizing force controller 42. Thus, the pressurizing force controller 42 can grasp the time change of the weight of each of the bags and the time change of the weight of each of the solutions so that the operation of the pressurization means 41 can be adjusted by the pressurizing force controller 42.

Further, even if the time change of the weight of each of the solutions is not grasped, the concentration rate of the concentrated solution can be adjusted by controlling the weight of the concentrated solution bag CB and the weight of the waste solution bag DB at a predetermined ratio (for example, 1:9).

Note that the method for measuring the weight of each of the bags is not particularly limited. In general, a method for measuring a weight of an object can be employed. For example, in the case where a bag is held in a state in which the bag hangs down, the weight can be measured using a suspension scale. Further, in the case where the bag placed on a table is pressurized, the weight can be measured using a usual platform scale.

When the weight of the concentrated solution and the weight of water can be grasped, the discharge flow rate and the flow rate of the concentrated solution is calculable and further the supply flow rate is calculable. Accordingly, it is not necessary to measure the weight of the stock solution bag UB. However, the measurement of the weight of the stock solution bag UB results in advantages of making the calculation accuracy of the supply flow rate higher and estimating the weight of cells captured by the filter.

The above example has described that the flow rate of the solution and the weight of the bag are used in the case where the supply flow rate is adjusted by controlling the operation of the pressurization means 41 (the case of the stock solution concentrating device 1C). In addition, in the case where the supply flow rate, the flow rate of the concentrated solution, and the flow rate of water are adjusted by a pump or clamp (in the case of the stock solution concentrating devices 1A, 1B, and 1C), it is certainly possible to control the operation of the pump or clamp by using the flow rate of the solution and the weight of the bag.

### (Stock Solution Treatment Device of Embodiment)

Subsequently, the stock solution treatment device of the embodiment will be described.

The stock solution treatment device of the embodiment includes a recovery unit that recovers the stock solution. The stock solution supplied from the recovery unit to the stock solution concentrating device is filtered and concentrated to form a concentrated solution. In the stock solution treatment device of the embodiment, it is configured that, while continuously performing the treatment of the stock solution, the concentrated solution can be administered to a patient simultaneously with the treatment of the stock solution.

Note that, in the stock solution treatment device of the embodiment, the stock solution concentrating device 1 is employed as a stock solution concentrating device, and thus hereinafter, the description about the stock solution concentrating device 1 will be omitted, if appropriate. Hereinafter, the stock solution concentrating device 1 (refer to FIG. 1) which has the same basic configuration as that of the stock solution concentrating device 1A will be typically described.

In FIG. 5, a symbol 50 denotes a recovery unit. The recovery unit 50 has a function that recovers a stock solution such as pleural and ascitic fluid and stores the stock solution. The recovery unit 50 has a function that stores the stock solution and supplies the stored stock solution to the stock solution concentrating device 1A. As the recovery unit 50, a usable example is a unit including a container that stores the stock solution and a transportation means that transports the stock solution from the container to the outside, such as a pump.

The recovery unit 50 is communicated with the stock solution bag UB of the stock solution concentrating device 1A through a tube 50a so that the stock solution stored in the container can be supplied to the stock solution bag UB by the transportation means.

Note that the recovery unit 50 may supply the pleural and ascitic fluid to the stock solution bag UB of the stock solution concentrating device 1A as described above.

Further, the pleural and ascitic fluid may be directly supplied to the tube 2 connected to the supply port 10a of the filter 10. In this case, the container of the recovery unit 50 substantially serves as the stock solution bag UB of the stock solution concentrating device 1A.

In the stock solution treatment device of the embodiment, the stock solution recovered in the recovery unit 50 is treated by the stock solution concentrating device 1A. The stock solution concentrating device 1A comprises the tube 4 that discharge the concentrated solution from the concentrator 20 (corresponding to the concentrated solution recovery pipe, which is described in the claims) including a plurality of branch pipelines 4a. The concentrated solution bags CB can be detachably attached to each of the branch pipelines 4a. Further, a flow rate adjusting member that stops the flow of the solution in each of the branch pipelines 4a or changes the flow rate, such as a clamp, may be provided at the branch pipelines 4a. In other words, the flow rate adjusting member is configured to be capable of establishing or blocking the communication between each of the concentrated solution bags CB and the concentrator 20.

For this reason, the concentrated solution bags CB communicated with the concentrator 20 are sequentially switched, whereby it is possible to adjust such that when a predetermined amount of the concentrated solution is stored in one concentrated solution bag CB, the concentrated solution is supplied to the other concentrated solution bag CB. Thus, during the use of one concentrated solution bag CB for drip infusion, the concentrated solution can be stored in the other concentrated solution bag CB. Accordingly, the concentrated solution can be administered to a patient while continuously performing the treatment of the stock solution. In other words, the treatment of the stock solution can be performed in parallel to the administration of the concentrated solution. Consequently, it is possible to reduce the time required for the treatment of administering the concentrated water after the treatment to the patient while treating the pleural and ascitic fluid taken from the patient as the stock solution (cell-free and concentrated ascites reinfusion therapy).

Note that, in the stock solution treatment device of the embodiment, it is not necessary to always perform the treatment of the pleural and ascitic fluid continuously. For example, during the cell-free and concentrated ascites reinfusion therapy, when the concentrated solution can be formed so as to keep the drip infusion of the concentrated solution, the treatment of the pleural and ascitic fluid may be performed intermittently. In other words, when no pleural and ascitic fluid remains in the stock solution bag UB, the treatment is once stopped. When the amount of the concentrated solution is less than a predetermined amount, the treatment is again started.

However, when the concentrated solution can be continuously formed during the cell-free and concentrated ascites reinfusion therapy, it is possible to reduce the burden of an operator who performs the cell-free and concentrated ascites reinfusion therapy. Hence, in the case of performing intermittent operation, when the treatment is started, it is necessary to perform the operation for supplying the pleural and ascitic fluid to the stock solution bag UB. On the other hand, when the stock solution is supplied from the recovery unit 50 so that the stock solution bag UB always contains a constant amount or more of the pleural and ascitic fluid, the operator does not need to supply the stock solution to the stock solution bag UB. This can reduce the burden of the operator.

Examples of the recovery unit 50 that maintains the above state include a recovery unit having a container of a volume capable of storing the total amount of the pleural and ascitic fluid recovered from a patient and a recovery unit in which a plurality of containers for storing the pleural and ascitic fluid recovered from a patient is provided and containers for supplying the stock solution to the stock solution bag UB are sequentially switched.

In the former case, when the pleural and ascitic fluid recovered from the patient is stored in a container and the pleural and ascitic fluid is supplied from the container to the stock solution bag UB by the transportation means, the pleural and ascitic fluid can be continuously supplied to the stock solution bag UB.

In the latter case, a container for storing the pleural and ascitic fluid recovered from the patient and a container for supplying the pleural and ascitic fluid to the stock solution bag UB are separately provided so that the pleural and ascitic fluid can be continuously supplied to the stock solution bag UB.

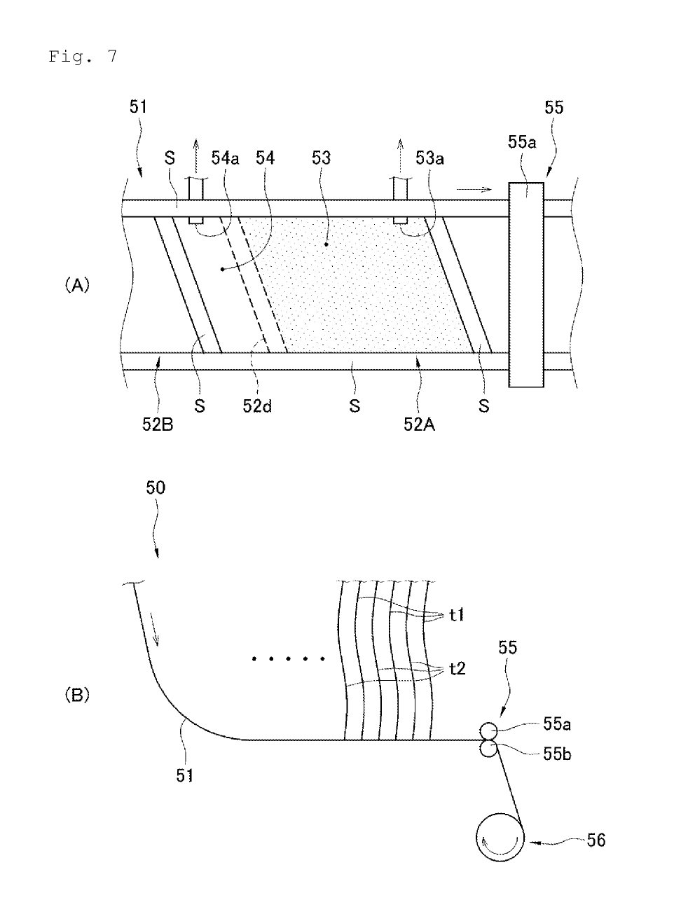

Further, the configuration to achieve the latter case (i.e., in the case where the recovery unit 50 has a plurality of containers) is not particularly limited. For example, the configuration as shown in FIG. 7 may be used.

In FIG. 7, a symbol 51 denotes a recovery sheet. The recovery sheet 51 is, for example, a long sheet formed into a stripe shape. The recovery sheet 51 is formed so as to have a plurality of recovery chambers 52 (corresponding to the above containers) therein.

For example, two long sheets are overlapped with each other and the ends are bonded together in the width direction at appropriate intervals in the longitudinal direction, whereby the recovery sheet 51 having the recovery chambers 52 can be formed.

Alternatively, long sheets are folded up so that the ends in the width direction are overlapped with each other and the overlapped ends are bonded together at appropriate intervals in the longitudinal direction, whereby the recovery sheet 51 having the recovery chambers 52 can be formed.

As shown in FIG. 7, one end of the long recovery sheet 51 is connected to a winding unit 56. When the winding unit 56 is operated, the sheet is wound into a roll shape. The structure of the winding unit 56 is not particularly limited. For example, a structure comprising a roll rotated by a motor and having a mechanism capable of fixing one end of the recovery sheet 51 on the roll can be employed as the winding unit 56.

As shown in FIG. 7, a pressurization unit 55 is provided at the upstream of the winding unit 56. The pressurization unit 55 comprises a pair of rolls 55a and 55b which is disposed in a state where the recovery sheet 51 is inserted between the rolls 55a and 55b. The rolls 55a and 55b are formed such that a space between both the rolls 55a and 55b is approximately equal to or slightly wider than the thickness of the recovery sheet 51.

For this reason, when the winding unit 56 is operated, the recovery sheet 51 is passed through between the rolls 55a and 55b of the pressurization unit 55 and wound by the winding unit 56. Thus, the storage of the pleural and ascitic fluid in each of the recovery chambers 52 of the recovery sheet 51 enables the pleural and ascitic fluid to be slightly pressurized when passing the recovery sheet through between the rolls 55a and 55b.

Here, as shown in FIG. 7, a stock solution supply port 53a and a stock solution discharge port 54a aligned at certain intervals in the longitudinal direction are provided in each of the recovery chambers 52 of the recovery sheet 51.

The stock solution supply port 53a is provided at the downstream of each of the recovery chambers 52 (at the side of the winding unit 56). The stock solution supply port 53a of each of the recovery chambers 52 is communicated with one end of each pipeline t1. On the other hand, the other end of each of the pipelines t1 is connected to each tube for discharging the pleural and ascitic fluid from the patient. A supply flow rate adjusting member that stops the flow of the solution in each of the pipelines t1 or changes the flow rate is provided on each of the pipelines t1. The flow rate adjusting member is configured to be capable of establishing or blocking the communication between the tube for discharging pleural and ascitic fluid and each of the recovery chambers 52. For example, the supply flow rate adjusting member can control the flow of the pleural and ascitic fluid such that the pleural and ascitic fluid is supplied in order, from the recovery chamber 52 located at the downstream.

Further, the stock solution discharge port 54a is provided at the upstream from the stock solution supply port 53a in each of the recovery chambers 52. The stock solution discharge port 54a of each of the recovery chambers 52 is communicated with one end of respective pipelines t2. On the other hand, the other end of each of the pipelines t2 is connected to each tube for discharging the pleural and ascitic fluid from the patient.

In each of the recovery chambers 52, a separation part 52d that divides a recovery chamber 52 into two chambers (a front chamber 53 and a rear chamber 54) is provided in the longitudinal direction of the recovery sheet 51. Specifically, the separation part 52d is provided so as to separate each of the recovery chambers 52 into a chamber (the front chamber 53) that is communicated with the stock solution supply port 53a and a chamber (the rear chamber 54) that is communicated with the stock solution discharge port 54a.

The separation part 52d is provided so as to communicate the front chamber 53 with the rear chamber 54 when the pleural and ascitic fluid in the front chamber 53 is pressurized in a state where the pleural and ascitic fluid is stored in the front chamber 53. For example, the separation part 52d is configured to have a weak sealed structure (bonding with a weak bonding strength). When the pleural and ascitic fluid in the front chamber 53 is pressurized, the force is added so as to expand the front chamber 53. Then, the seal is broken, which allows the front chamber 53 to be communicated with the rear chamber 54.

Note that the separation part 52d may have any structure as long as it enables the front chamber 53 to be communicated with the rear chamber 54 when the pleural and ascitic fluid in the front chamber 53 is pressurized.

Since the recovery unit 50 has such a structure described above, the pleural and ascitic fluid recovered from the patient can be continuously supplied to the stock solution bag UB or the tube 2 when the recovery sheet 51 is wound by the winding unit 56.

In other words, the pleural and ascitic fluid recovered from the patient is passed through each of the pipelines t1 and supplied to the front chamber 53 of the recovery chamber 52 (a first recovery chamber 52A) located at the downstream in the recovery sheet 51. Then, when a certain amount of pleural and ascitic fluid is supplied to the front chamber 53 of the first recovery chamber 52A, the supply flow rate adjusting member stops the pleural and ascitic fluid from being supplied to the first recovery chamber 52A. Then, the pleural and ascitic fluid is supplied to the front chamber 53 of the recovery chamber 52 (a second recovery chamber 52B) located at the downstream of the first recovery chamber 52A.

On the other hand, when the recovery sheet 51 is wound by the winding unit 56, the first recovery chamber 52A is transferred to the pressurization unit 55 and eventually drawn between the rolls 55a and 55b.

When the first recovery chamber 52A is drawn between the rolls 55a and 55b of the pressurization unit 55, the pleural and ascitic fluid in the front chamber 53 is pressurized. Then, the seal of the separation part 52d is broken, and the front chamber 53 is communicated with the rear chamber 54. As a result, the pleural and ascitic fluid flows from the front chamber 53 to the rear chamber 54. Thereafter, the pleural and ascitic fluid, which has been flowed into the rear chamber 54 by the pressurizing force applied to the pleural and ascitic fluid in the front chamber 53, flows into the stock solution bag UB or the tube 2 through the stock solution discharge port 54a and each of the pipelines t2. In other words, the pleural and ascitic fluid can be supplied from the recovery unit 50 to the stock solution bag UB or the tube 2 by the pressurizing force of the pressurization unit 55.

Additionally, the pleural and ascitic fluid is sequentially supplied to the recovery chamber 52 at the downstream. The front chamber 53 is sequentially filled with the pleural and ascitic fluid. Then, each of the recovery chambers 52 is drawn between the roll 55a and 55b of the pressurization unit 55, sequentially in order from the recovery chamber 52 at the downstream so that the pleural and ascitic fluid can be sequentially supplied from the rear chamber 54 of the recovery chamber 52 at the downstream to the stock solution bag UB or the tube 2. In other words, the pleural and ascitic fluid can be continuously supplied from the recovery unit 50 to the stock solution bag UB or the tube 2.

Note that, in the case where the front chamber 53 is pressurized by the rolls 55a and 55b of the pressurization unit 55, the pleural and ascitic fluid may flow back from each of the pipelines t1 into the patient through the stock solution supply port 53a. However, when the supply flow rate adjusting member stops the pleural and ascitic fluid from being supplied to the recovery chamber 52, it is possible to prevent the pleural and ascitic fluid from flowing back into the patient.

Further, even when a check valve is provided at each of the pipelines t1 or the stock solution supply port 53a, it is possible to prevent the pleural and ascitic fluid from flowing back into the patient.

Further, in the case of using a resin tube as each of the pipelines t1, the back flow can be prevented by cutting off and sealing each of the pipelines t1 after a predetermined amount of pleural and ascitic fluid is supplied to the front chamber 53. In the case of this configuration, when the recovery chamber 52 of the recovery sheet 51 is transferred to a predetermined position, each of the pipelines t1 connected to the recovery chamber 52 may be automatically cut off and sealed.

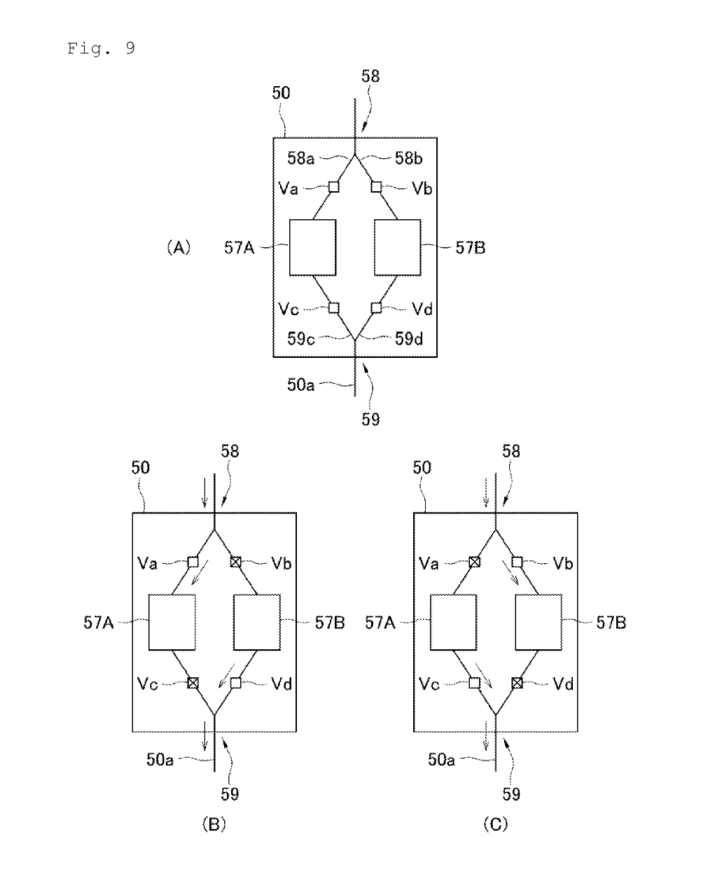

Further, as the recovery unit 50a in which a plurality of containers for storing the pleural and ascitic fluid recovered from the patient is provided and containers for supplying the stock solution to the stock solution bag UB are switched, the configuration of FIG. 9 can be used, in addition to the configuration of FIG. 7.

As shown in FIG. 9, a pair of bags 57A and 57B is provided as the recovery unit 50. A Y-shaped pipeline 58 is provided as a communication passage that connects the tube for discharging the pleural and ascitic fluid from the patient to the bags 57A and 57B. On the other hand, a Y-shaped pipeline 59 is provided as a communication passage that connects the bags 57A and 57B to the stock solution bag UB.

In the pipeline 58, valves va and vb for establishing or blocking the communication between branch paths 58a and 58b are provided on the branch paths 58a and 58b communicated with the bags 57A and 57B respectively.

On the other hand, in the pipeline 59, valves vc and vd for establishing or blocking the communication between branch paths 59c and 59d are provided on the branch paths 59c and 59d communicated with the bags 57A and 57B respectively.

Additionally, when the valve va is opened, the valve vd is opened and controls the valves vb and vc to be closed (refer to operation A and FIG. 9 (B)). When the valve vb is opened, the valve vc is opened and controls the valves va and vd to be closed (refer to operation B and FIG. 9 (C)).

According to the configuration, the pleural and ascitic fluid in the bag 57B can be supplied to the stock solution bag UB while collecting the pleural and ascitic fluid discharged from the patient in the bag 57A in the operation A (refer to FIG. 9 (B)). Further, the pleural and ascitic fluid in the bag 57A can be supplied to the stock solution bag UB while collecting the pleural and ascitic fluid discharged from the patient in the bag 57B in the operation B (refer to FIG. 9 (C)). In other words, the switching between the operation A and the operation B allows for simultaneous performance of the recovery of the pleural and ascitic fluid discharged from the patient and the supply of the recovered pleural and ascitic fluid to the stock solution bag UB. Additionally, when the timing of switching between the operation A and the operation B is adjusted, it is possible to continuously perform the recovery of the pleural and ascitic fluid and the supply of the pleural and ascitic fluid to the stock solution bag UB. The bag 57 communicated with the stock solution bag UB is always maintained in a state of being separated from the patient (the bag 57 is not communicated with the patient's peritoneal cavity). Therefore, even if the recovery of the pleural and ascitic fluid discharged from the patient and the supply of the pleural and ascitic fluid in the bag 57 to the stock solution bag UB are continuously performed, there is no influence on the patient.

Each of the Y-shaped pipelines 58 and 59 or each of the valves va to vd corresponds to the flow path adjusting mechanism, which is described in the claims. Note that, the flow path adjusting mechanism is not limited to the configuration described above, and any configuration exhibiting the function can be employed.

### (Continuous Treatment)

In the case of performing the cell-free and concentrated ascites reinfusion therapy using the stock solution treatment device of the embodiment, the treatment time can be reduced significantly, compared to the conventional method.

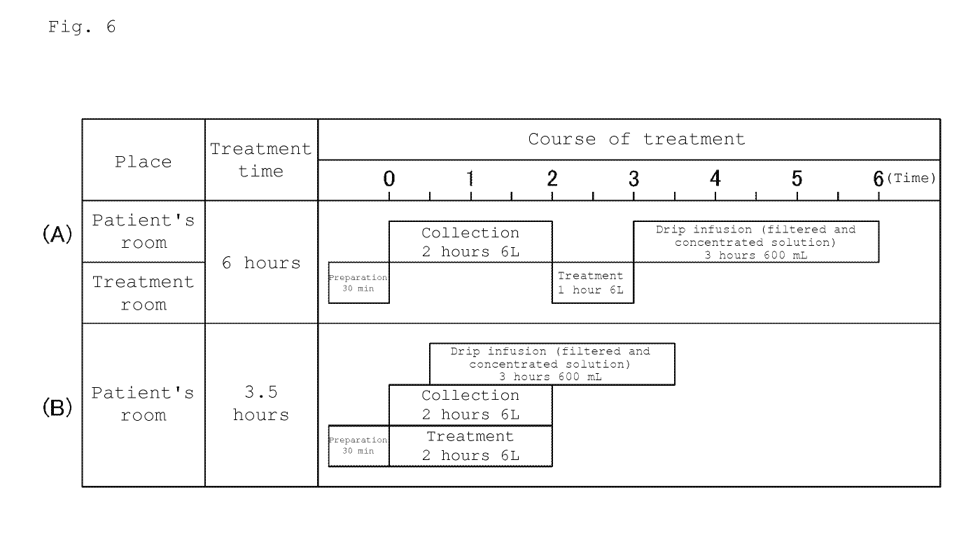

As shown in FIG. 6, in the conventional cell-free and concentrated ascites reinfusion therapy (FIG. 6 (A)), the preparations for the treatment of the pleural and ascitic fluid are first performed in a treatment room. The time required for the preparations is about 30 minute.

When the preparations for the treatment are completed, the pleural and ascitic fluid is recovered from the patient. For example, the time required for recovering 6 L of the pleural and ascitic fluid is about 2 hours. When the speed for recovering the pleural and ascitic fluid is increased, the time required for recovery is decreased. However, water in the blood is converted to the pleural and ascitic fluid. This reduces the amount of water and useful components in the blood. Thus, these components need to be administered via drip infusion. Hence, regardless the fact that it is a treatment for reducing the amount of pharmaceutical products by returning a concentrated solution of the useful components contained in the pleural and ascitic fluid to the patient, pharmaceutical products as useful components need to be supplied when recovering the pleural and ascitic fluid. As a result, the intended effect of the cell-free and concentrated ascites reinfusion therapy (a reduction in the amount of pharmaceutical products) is not given. Accordingly, the time required to collect the pleural and ascitic fluid is long to some extent.

Since the concentrated solution is returned to the patient via drip infusion, it is not possible to return the solution at a constant speed or more. In the case of collecting 6 L of the pleural and ascitic fluid, the amount of the concentrated solution is about 600 ml. It takes about 3 hours to return the amount of the concentrated solution.

Therefore, in the conventional cell-free and concentrated ascites reinfusion therapy, the time required to collect 6 L of the pleural and ascitic fluid and to return the concentrated solution to the patient is about 6 hours, excluding the preparation time.

On the other hand, in the case of the stock solution treatment device of the embodiment (refer to FIG. 6 (B)), the concentration treatment can be performed almost simultaneously with the collection of the pleural and ascitic fluid. While treating the pleural and ascitic fluid to form a concentrated solution, a part of the concentrated solution can be returned to the patient. Therefore, the time required to collect 6 L of the pleural and ascitic fluid and to return the concentrated solution to the patient can be reduced to about half of that of the conventional cell-free and concentrated ascites reinfusion therapy (3.5 hours).

Thus, although the conventional cell-free and concentrated ascites reinfusion therapy makes hospitalization necessary, the above condition makes outpatient treatment possible. This results in improvements in the quality of life (QOL) and convenience of treatment for patients.

For example, in the case where the amount of the pleural and ascitic fluid is 6 L, it takes about 20 minutes to collect 1 L of the pleural and ascitic fluid. This level of collection speed can reduce the burden on the patient.

In the case of the concentration treatment of 1 L of the pleural and ascitic fluid, the treatment can be completed in about 10 minutes. Thus, the drip infusion of the concentrated solution can be started 30 minutes at the latest after the collection of the pleural and ascitic fluid has started. Of course, when the concentration treatment is performed in parallel with the collection of the pleural and ascitic fluid, the drip infusion can be started further earlier.

Additionally, in the case of the stock solution treatment device of the embodiment, it is possible to significantly reduce the time required to store the pleural and ascitic fluid collected from the patient and the concentrated solution. This hardly causes problems such as contamination of the pleural and ascitic fluid and the concentrated solution, and allows the treatment to be safely carried out.

### (Circulation Treatment Device)

The stock solution treatment device of the embodiment is configured such that, in the case where the pleural and ascitic fluid discharged from a patient is returned to the patient, the concentrated solution is once stored in a concentrated solution bag CB, the concentrated solution bag CB is detached from the stock solution concentrating device 1, and the solution is returned to the patient by a method such as drip infusion.

However, the concentrated solution may be directly returned to the patient through the tube 4 that discharges the concentrated solution from the concentrator 20 of the stock solution concentrating device 1 or a tube (return tube) that is communicated with the concentrated solution bag CB.

Thus, while continuously treating the stock solution, the concentrated solution can be returned to the patient. Once the device is set up, the concentrated solution can be continuously returned to the patient until no stock solution remains. As a result, it is unnecessary to replace the concentrated solution bag CB. This can decrease the frequency of checking the treatment state and reduce the burden of the operator who performs the treatment.

As described above, in the case where the concentrated solution is directly returned from the concentrator 20 of the stock solution concentrating device 1 of the stock solution treatment device of the embodiment to the patient, the pleural and ascitic fluid from the thoracic and peritoneal cavities of a patient may be directly recovered in a recovery unit through a recovery tube. Then, it is possible to form an extracorporeal circulation loop connecting the thoracic and peritoneal cavities to veins of the patient through the recovery tube, the recovery unit, the filter 10 of the stock solution concentrating device 1, the concentrator 20, the concentrated solution bag CB, and the return tube. In other words, it is possible to form a circulation loop for treating the pleural and ascitic fluid discharged from the thoracic and peritoneal cavities of the patient by the stock solution treatment device to form a concentrated solution, and returning the concentrated solution to the patient.

When the circulation loop is formed, the concentrated solution can be returned to the patient while continuously treating the pleural and ascitic fluid. Once the device is set up, the pleural and ascitic fluid can be continuously recovered until no pleural and ascitic fluid to be treated remains. Additionally, the recovered pleural and ascitic fluid can be continuously treated and the concentrated solution can be continuously returned to the patient. As a result, it is unnecessary to replace the stock solution bag UB or the concentrated solution bag CB. This can decrease the frequency of checking the treatment state and reduce the burden of the operator who performs the treatment.

The method for forming a circulation loop, namely a method for forming a flow path for flowing the pleural and ascitic fluid or the concentrated solution, is not particularly limited. Various methods and devices can be used.

For example, a tube (a recovery tube) having a needle for dehydration formed on one end thereof is inserted into the thoracic or peritoneal cavity of the patient, and the other end of the recovery tube is directly connected to the stock solution bag UB. After that, the pleural and ascitic fluid recovered through the recovery tube is supplied to the filter via the stock solution bag UB. This allows the pleural and ascitic fluid to be directly and continuously treated by the stock solution concentrating device 1. Then, the obtained concentrated water is directly returned from the concentrated solution bag CB or the tube 4 of the stock solution concentrating device 1 to the patient through the return tube so that the circulation loop can be formed.

Note that, in the case of the configuration, the stock solution bag UB serves as the reservoir of the recovery unit, which is described in the claims.

Of course, the recovered pleural and ascitic fluid may be stored in a reservoir in which the recovered pleural and ascitic fluid is once stored (for example, a bag or plastic case, such as an infusion solution bag), separately from the stock solution bag UB. In this case, a pump or gravity is used to continuously supply the pleural and ascitic fluid from the reservoir to the stock solution bag UB or the tube 2. This allows the pleural and ascitic fluid to be continuously treated by the stock solution concentrating device 1.

The stock solution treatment device of the embodiment as configured above corresponds to the circulation treatment device, which is described in the claims. In other words, the recovery unit of the stock solution treatment device of the embodiment corresponds to the recovery unit of the circulation treatment device, which is described in the claims. The recovery unit and the concentration unit of the stock solution treatment device of the embodiment each respectively corresponds to the treatment unit of the circulation treatment device, which is described in the claims. Further, the return tube corresponds to the return unit of the circulation treatment device, which is described in the claims.

Of course, needless to say, as the circulation treatment device of the present invention, it is also possible to employ the configurations other than the stock solution treatment device of the embodiment. Specifically, it is possible to employ a concentration device other than the stock solution concentrating device 1 of the present invention, for example, a device for transporting a stock solution (pleural and ascitic fluid) under conditions where negative pressure is generated, and performing filtration and concentration. In this case, the recovery unit of the circulation treatment device of the present invention once stores the recovered pleural and ascitic fluid in the reservoir. This can prevent the influence of the negative pressure on the recovery tube for recovering the pleural and ascitic fluid from the patient. In other words, even if the device for transporting a stock solution under conditions where negative pressure is generated, and performing filtration and concentration is used as the treatment unit, it is possible to prevent the influence of the negative pressure on the patient. Thus, even in the case where the extracorporeal circulation of the pleural and ascitic fluid is performed and the treatment solution such as a concentrated solution is returned to the patient, the treatment can be safely performed.

For example, in the case of using the recovery unit 50 as shown in FIG. 7, while the stock solution is recovered in the recovery chambers 52A and 52B, the treatment unit is separated from the recovery tube by the separation part 52d. Accordingly, even if negative pressure is generated in the treatment unit, it is possible to prevent the influence of the negative pressure on the patient via the recovery tube.

Similarly, even in the case of using the recovery unit 50 as shown in FIG. 9, the bag 57 communicated with the stock solution bag UB is always maintained in a state of being separated from the patient. Accordingly, even if negative pressure is generated in the treatment unit, it is possible to prevent the influence of the negative pressure on the patient via the recovery tube.

Note that the circulation treatment device of the present invention is configured such that the conditions of the treatment unit do not affect the patient. On a recovery tube that allows a patient and a reservoir to communicate with each other, a sensor that detects a state of the stock solution flowing in the recovery tube is provided just in case so that the treatment can be safely performed. For example, when a sensor capable of detecting the flow rate of the stock solution flowing in the recovery tube and the pressure of the recovery tube is provided, it is possible to detect that the discharge of the pleural and ascitic fluid is completed, the speed of the discharge of the pleural and ascitic fluid is more than necessary, and negative pressure is generated in the recovery tube. Then, when the operation conditions of the device are changed or the operation is stopped based on a signal from the sensor, it is possible to prevent the negative pressure from being applied to the patient in some way. Further, it is possible to prevent an unnecessary burden from being applied to the patient. Further, when the sensor detects abnormalities (a decrease in the flow rate of the pleural and ascitic fluid, generation of negative pressure or the like), the sensor gives an alarm for the operator based on a signal. Consequently, the operator does not have to always check the device.

Note that, the sensor to be provided on the recovery tube is, for example, a pyro sensor (a sensor that mechanically detects the thickness of a tube and determines a reduction in flow rate based on changes in the thickness) or an ultrasonic sensor, however, it is not particularly limited thereto.

Alternatively, instead of providing a recovery tube, a sensor is provided on a reservoir in order to grasp the state of the stock solution flowing from the patient into the reservoir through the recovery tube or the conditions of the thoracic and peritoneal cavities of the patient. For example, in the case of using a sac-like member as the reservoir, a sensor for measuring the thickness or even the pyro sensor provided on the reservoir can grasp the state of the stock solution flowing into the reservoir.

The recovery unit and the stock solution concentrating device 1 constituting the circulation-type treatment device may be connected when used. This leads to an advantage such that the recovery unit and the stock solution concentrating device 1 can be changed appropriately depending on the application purpose.

On the other hand, in the case of connecting the recovery unit or the stock solution concentrating device 1, it is necessary to have a certain degree of knowledge and a certain amount of experience. If a less experienced and informed person (for example, a patient or patient's relatives) is able to easily perform the treatment, it is possible to perform the treatment in the patient's house. Further, the treatment can be easily performed only in the patient's room in the hospital. In addition to the advantage of decreasing the treatment time, which has been described regarding the stock solution treatment device, the treatment may be performed at any place. This improves the convenience of treatment for patients and reduces the burden on patients.

For example, each of the containers of the stock solution concentrating device 1 and the reservoir of the recovery unit are housed in a case like a suitcase. Besides, each of the containers is connected by the tubes. Thus, the treatment for filtering and concentrating the pleural and ascitic fluid and returning the concentrated solution to the patient can be performed by only preparing the case.

Particularly, all of the recovery bag of the recovery unit, the concentrated solution bag CB, and the waste solution bag DB are housed in the case. Then, the treatment can be performed by connecting the recovery tube or the return tube to the patient. Thus, the treatment can be carried out very simply in a shorter time. In this case, when a lid of the case is opened, a cartridge used for the filter 10 or the concentrator 20 can be attached or detached. Particularly, when the attaching and detaching operation can be performed with just one touch, the maintenance is made easy.

Alternatively, the filter 10 and the concentrator 20 are installed in the case. Then, the recovery bag, the concentrated solution bag CB, and the waste solution bag DB are separately prepared. After that, these components may be connected.

Further, the case may be configured to be closed for use in performing the treatment. Or, the case may be configured to be opened for use in performing the treatment. The configuration is not particularly limited. The case is opened for use in performing the treatment, whereby there is yielded an advantage of easily confirming the treated state with the naked eyes.

Further, it may be configured such that, like a general carrying case, an extendable handle is provided so that each of the bags can be hung on this handle. This is advantageous such that it is not necessary to prepare a member for hanging each of the bags, and that another container for drip infusion can be attached to the handle.

### INDUSTRIAL APPLICABILITY

The stock solution concentrating device of the present invention is suitable as a device for filtering and concentrating the pleural and ascitic fluid containing cells, the blood during surgery or the blood during bloodletting to form a concentrated solution or a device for purifying the plasma waste fluid discarded in plasma exchange and reusing each of them.

The stock solution treatment device of the present invention is suitable as a device for continuously recovering the pleural and ascitic fluid containing cells, the blood during surgery or the blood during bloodletting and continuously treating each of them.

### DESCRIPTION OF REFERENCE SIGNS

1 Stock solution concentrating device
10 Filter
20 Concentrator
30 Circulation flow path
31 Circulation flow forming means
36 Nozzle
40 Pressurization mechanism
50 Recovery unit
51 Recovery sheet
52A Recovery chamber
52B Recovery chamber
52d Separation part
53 Front chamber
53a Supply port
54 Rear chamber
54a Supply port
55 Pressurization unit
56 Winding unit
57A Bag
57B Bag
UB Stock solution bag
CB Concentrated solution bag
DB Waste solution bag

1-21. (canceled)

22. A stock solution concentrating device for concentrating a stock solution such as pleural and ascitic fluid or blood plasma to form a concentrated solution, the device comprising:
a filter having a filtration member that filters the stock solution;
a concentrator to which a filtrate which has been filtered by the filter is supplied, and which concentrates the filtrate to form the concentrated solution; and
a stock solution supply unit that supplies the stock solution to the filter,
wherein the stock solution supply unit has a supply amount adjustment function for adjusting the amount of the stock solution supplied to the filter,
the filter includes
a supply port to which the stock solution is supplied, and
a separate liquid discharge port from which a separate liquid which has been separated from the filtrate is discharged,
the stock solution supply unit includes
a flow path that supplies the stock solution to the supply port of the filter, and
a circulation flow path that supplies the separate liquid discharged from the separate liquid discharge port of the filter to the supply port of the filter, and
the circulation flow path is equipped with a circulation flow forming means that forms a flow of the separate liquid flowing from the separate liquid discharge port of the filter to the supply port of the filter.

23. A stock solution concentrating device for concentrating a stock solution such as pleural and ascitic fluid or blood plasma to form a concentrated solution, the device comprising:
a filter having a filtration member that filters the stock solution;
a concentrator to which a filtrate which has been filtered by the filter is supplied, and which concentrates the filtrate to form the concentrated solution; and
a stock solution supply unit that supplies the stock solution to the filter,
wherein the stock solution supply unit has a supply amount adjustment function for adjusting the amount of the stock solution supplied to the filter,
the filter includes
a supply port to which the stock solution is supplied, and
a separate liquid discharge port from which a separate liquid which has been separated from the filtrate is discharged,
the stock solution supply unit includes
a circulation flow path that supplies the separate liquid discharged from the separate liquid discharge port of the filter to the supply port of the filter, and
the circulation flow path is equipped with a circulation flow forming means that forms a flow of the separate liquid flowing from the separate liquid discharge port of the filter to the supply port of the filter,
the circulation flow forming means includes
a nozzle whose end is connected to the circulation flow path and
a stock solution supply means that supplies the stock solution to the nozzle,
the end of the nozzle is connected to the circulation flow path so that the stock solution is supplied from the end of the nozzle to the circulation flow path when the stock solution is supplied from the stock solution supply means, and
the nozzle is disposed so that a flow of the stock solution having a speed component in a direction flowing from the separate liquid discharge port of the filter to the supply port of the filter is formed by the stock solution supplied from the end of the nozzle to the circulation flow path.

24. The stock solution concentrating device according to claim 22,
wherein the stock solution supply unit includes
a flow generating means that forms a flow flowing from the filter to the concentrator on a flow path that allows the filter to be communicated with the concentrator.

25. The stock solution concentrating device according to claim 22,
wherein the stock solution supply unit includes a stock solution storage container that stores the stock solution, and
the circulation flow path is provided in order to return the separate liquid discharged from the separate liquid discharge port of the filter to the stock solution storage container.

26. The stock solution concentrating device according to claim 23,
wherein the stock solution supply means includes
a stock solution storage part for storing the stock solution that is communicated with the nozzle and
a pressurization mechanism that pressurizes the stock solution in the stock solution storage part.

27. The stock solution concentrating device according to claim 26 comprising a water removing means that discharges water separated from the filtrate from the concentrator at a predetermined flow rate,
wherein the pressurization mechanism includes a pressurizing force controller that detects a flow rate of the concentrated solution discharged from the concentrator and adjusts the pressurizing force pressurizing the stock solution based on the flow rate of the concentrated solution.

28. The stock solution concentrating device according to claim 26 comprising a concentrated solution discharge means that discharges the concentrated solution from the concentrator at a predetermined flow rate,
wherein the pressurization mechanism includes a pressurizing force controller that detects the flow rate of water separated from the filtrate discharged from the concentrator and adjusts the pressurizing force pressurizing the stock solution based on the flow rate of the water.

29. The stock solution concentrating device according to claim 26,
wherein the stock solution supply means includes a weight measurement function that measures the weight of the stock solution stored in the stock solution storage part,
the pressurization mechanism includes
a water weight measurement unit that measures the weight of water separated from the filtrate and
a concentrated solution weight measurement unit that measures the weight of the concentrated solution discharged from the concentrator, and
the pressurization mechanism includes a pressurizing force controller that adjusts the pressurizing force pressurizing the stock solution based on all or any of the weight of the stock solution measured by the weight measurement function of the stock solution supply means, the weight of the concentrated solution measured by the concentrated solution weight measurement unit, and the weight of the water measured by the water weight measurement unit.

30. The stock solution concentrating device according to claim 22,
wherein a separated product recovery unit that discharges a liquid and/or residue in the circulation flow path and the filter is provided on the circulation flow path.

31. The stock solution concentrating device according to claim 22,
wherein the concentrator includes a concentrated solution recovery pipe that is connected to the separate liquid discharge port, and
the concentrated solution recovery pipe includes a plurality of branch pipelines to which concentrated solution storage containers for storing a concentrated solution are attached.

32. The stock solution concentrating device according to claim 31,
wherein the plurality of branch pipelines are provided so as to form a difference in height of the concentrated solution storage containers.

33. The stock solution concentrating device according to claim 31,
wherein a flow rate adjusting members that be capable of establishing or blocking the communication between each of the concentrated solution bags and the concentrator is provided on the plurality of branch pipelines.

34. The stock solution concentrating device according to claim 22, comprising
a heating means that heats the stock solution and/or the filtrate.

35. A stock solution treatment device that recovers a stock solution such as pleural and ascitic fluid or blood plasma,
the device comprising:
the stock solution concentrating device according to claim 22; and
a recovery unit that recovers a stock solution and supplies the recovered stock solution to the filter of the ascitic fluid concentration device,
wherein the concentrator of the stock solution concentrating device includes
a concentrated solution recovery pipe that is connected to the separate liquid discharge port, and
the concentrated solution recovery pipe includes
a plurality of branch pipelines to which concentrated solution storage containers for storing a concentrated solution are attached.

36. The stock solution treatment device according to claim 35,
wherein the recovery unit includes
a pair of bags for storing the stock solution, and further includes
a flow path adjusting mechanism that alternately supplies the stock solution to the pair of bags, and supplies the stock solution from one bag of the pair of bags to which a stock solution is not supplied, to the filter of the ascitic fluid concentration device.

37. The stock solution treatment device according to claim 35,
wherein the recovery unit includes
a recovery sheet having a plurality of recovery chambers isolated from each other,
in each of the recovery chambers of the recovery sheet,
a stock solution supply port for supplying a stock solution from the outside to the recovery chamber,
a stock solution discharge port for discharging the stock solution in the recovery chamber to the outside, and
a separation part that separates the inside of the recovery chamber into a front space communicated with the stock solution supply port and a rear space communicated with the stock solution discharge port are provided, and
the separation part is formed so that the front space is communicated with the rear space when the stock solution in the front space is pressurized in a state where the stock solution is stored in the front space.

38. The stock solution treatment device according to claim 37,
wherein the recovery unit includes
a winding unit that winds the recovery sheet and
a pressurization unit that is disposed at the upstream of the winding unit, and
the pressurization unit sequentially pressurizes the recovery sheet wound by the winding unit.

39. A circulation-type treatment device that recovers a stock solution such as pleural and ascitic fluid or blood plasma from the living body, performs an extracorporeal treatment, and returns the stock solution to the living body,
the device comprising:
a recovery unit that recovers the stock solution discharged from the living body;
a treatment unit that treats the stock solution recovered by the recovery unit; and
a return unit that returns the treatment solution treated by the treatment unit to the living body,
wherein the recovery unit includes a reservoir that once stores the stock solution discharged from the living body,
the treatment unit is the stock solution treatment device according to claim 35.

40. The circulation-type treatment device according to claim 39, wherein a recovery tube that allows the living body to be communicated with a reservoir is provided on the recovery unit, and
a sensor that detects a state of the stock solution flowing in the recovery tube is provided on the recovery tube.

41. The circulation-type treatment device according to claim 39, wherein the treatment unit and the return unit are connected to each other and stored in a case.
